(19) United States
(12) Patent Application Publication
(10) Pub. No.: US 2005/0164994 A1
(43) Pub. Date: Jul. 28, 2005

(54) Treatment of genitourinary tract disorders

(75) Inventors: Paul Ashton, Boston, MA (US); Grazyna Cynkowska, Brookline, MA (US); Tadeusz Cynkowski, Brookline, MA (US); Thomas J. Smith, Pasadena, CA (US)

Correspondence Address: FISH & NEAVE IP GROUP;ROPES & GRAY LLP, ONE INTERNATIONAL PLACE, BOSTON, MA, 02110-2624, US

(73) Assignee: Control Deliver Systems, Inc., Watertown, MA (US)

(21) Appl. No.: 10/859,902

(22) Filed: Jun. 3, 2004

Related U.S. Application Data
(63) Continuation-in-part of application No. 10/316,137, filed on Dec. 10, 2002.
(60) Provisional application No. 60/337,126, filed on Dec. 10, 2001.
(60) Provisional application No. 60/479,023, filed on Jun. 16, 2003.
(60) Provisional application No. 60/477,526, filed on Jun. 11, 2003.

(51) Int. Cl. A61K 31/60; A61K 31/58; A61K 9/22

(52) U.S. Cl. 514/159; 514/173; 604/890.1

(57) ABSTRACT

Genitourinary system disorders are treated with therapeutic agents, and optionally further with radiation treatments.

### CROSS-REFERENCE TO RELATED APPLICATIONS

This application is a continuation-in-part of U.S. patent application Ser. No. 10/316,137, filed Dec. 10, 2002, which claims the benefit of priority from U.S. Provisional Application No. 60/337,126, filed Dec. 10, 2001, and the specifications of which are incorporated by reference herein in their entirety. This application claims the benefit of priority from U.S. Provisional Application No. 60/477,526, filed Jun. 11, 2003 and from U.S. Provisional Application No. 60/479,023, filed Jun. 16, 2003, the specifications of which are incorporated by reference herein in their entirety.

### FIELD OF THE INVENTION

The present invention relates to the treatment of genitourinary tract disorders, and more particularly to treatment of disorders of the genitourinary tract by delivery of a therapeutic agent or agents.

### BACKGROUND OF THE INVENTION

Brachytherapy is radiation treatment based on implanted radioactive seeds emitting radiation from each seed. Careful pretreatment planning is done to assure proper placement of the radioactive seeds. However, there is often substantial and prolonged swelling of the prostate after brachytherapy. This swelling is responsible for some symptomatic side effects, and may also play a role in under- or over-treating the tumor. If prostatic swelling alters the configuration of the desired positions of the seeds, then certain areas may be under-treated, permitting the prostate cancer to survive, while other areas are over-treated causing increased side effects or even radiation injury. Since the swelling has an average half-life of 10 days, a significant amount of the life of the radiation seed is lost when the prostate is in a swollen state. This is more a concern when higher grade prostate cancer lesions are treated with palladium 103 (¹⁰³Pd), which has a relatively shorter half-life (17 days) than other lower-energy isotopes. The ¹⁰³Pd is used because of its higher energy release, which is believed to be more effective against more aggressive prostate tumors. However, if the seeds are not in the proper distribution, there is a potential for local failure. Further, the resultant swelling from the procedure and the inflammation from the radiation usually cause irritative voiding patterns as well as discomfort for several months. In a fair number of patients these symptoms can have a profound impact on their quality of life for an extended time period.

Surgical implantation often leads to other deleterious side effects such as pain and swelling. It is routine to treat surgical implant patients with systemic anti-inflammatory and analgesic drugs. As some post-operative patients experience fever, it is common to treat such patients with antipyretics. It is not uncommon for patients to show poor tolerance for systemic administration of certain NSAIDs, steroids, and opiates. Moreover, several NSAIDs act as blood thinners and anticoagulants, which may increase the risk of postoperative hemorrhage.

People also suffer from non-radiation treatment based genitourinary disorders, such as incontinence and prostatitis. Prostatitis, which is inflammation of the prostate, can be due very debilitating. Severe frequency, burning, pain and obstructive symptom are the most common complaints. The most common therapy for prostatitis is a combination of anti-inflammatory agents, antibiotics and alpha-blocking drugs to relax the intrinsic muscles of the prostate, which are often in spasm secondary to the inflammatory process.

Thus, devices for, and methods of, controlling inflammation, symptoms, and other side effects of brachytherapy, surgical implantation, and/or genitourinary disorders would be useful.

### SUMMARY OF THE INVENTION

One aspect of the invention provides a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device is dimensioned to position two radiation seeds a predetermined distance apart.

Another aspect of the invention provides a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties.

In certain embodiments, the first constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents.

In some embodiments, the second constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents.

In preferred embodiments, the first constituent moiety is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or a pharmaceutically acceptable salt thereof.

In some embodiments, the codrug has the structural formula:
R₁-L-(R₂)ₙ
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
n is an integer of from 1 to 4; and
L is selected from a direct bond and a linking group.

In other embodiments, the codrug has the structural formula:
R₁-(L-R₂)ₙ
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
n is an integer of from 1 to 4; and
L is selected from a direct bond and a linking group.

In some embodiments, the codrug has the structural formula:
(R₁-L)ₘR₂(L₂-R₃)ₙ
wherein the first constituent moiety is R₁;
the second constituent moiety is R₂;
R₁, R₂, and R₃ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
m is an integer of from 1 to 4;
n is an integer of from 1 to 4; and
L and L₂ are each independently selected a direct bond and a linking group.

In some embodiments, R₂ is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or a pharmaceutically acceptable salt thereof.

In certain embodiments, the first constituent moiety is a residue of alitretinoin (9-cis-retinoic acid); amifostine; bexarotene (4-[1-(5,6,7,8-tetrahydro-3,5,5,8,8-pentamethyl-2-naphthalenyl)ethenyl]benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-fluorouracil (5-FU); gemcitabine; hydroxyurea; idarubicin; irinotecan; melphalan; methotrexate; mitoxantrone; paclitaxel; pentostatin; streptozocin; temozolomide; teniposide; tomudex; topotecan; valrubicin (N-trifluoroacetyladriamycin-14-valerate); or vinorelbine.

In certain embodiments, the first constituent moiety is a residue of: 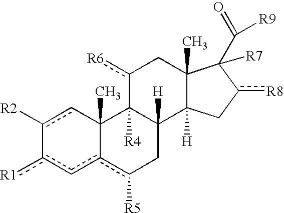
wherein R1 is ═O, —OH, or —(CH₂)₁₋₄Cl;
R2 is H, C₁₋₄alkyl, Cl, or Br;
R4 is H, F, or Cl;
R5 is H, F, Cl, CH₃, or —CHO;
R6 is H, OH, or Cl;
R7 is H, OH, CH₃, O—COCH₃, O(CO)OCH₂CH₃, O—(CO)-2-furanyl, or O—C(O)—(CH₂)₂CH₃;
R8 is H, CH₃, OH, ═CH₂, or together R7 and R8 form, together with the adjacent carbon atoms to which they are attached:

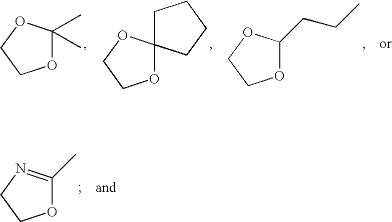

R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and
wherein the bonds indicated by 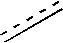
   are either double or single bonds.

In some embodiments, the first constituent moiety is residue of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide, or a pharmaceutically acceptable salt thereof.

In some embodiments, the drug delivery device further comprises a carrier, an excipient, a solvent, an adjuvant, a diluent, a dispersant, or a surfactant. In certain embodiments, the drug delivery device further comprises a biocompatible polymer such as polyvinyl alcohol (PVA).

In certain embodiments, the codrug or a pharmaceutically acceptable salt or prodrug thereof, is coated with the biocompatible polymer. In other embodiments, the codrug or a pharmaceutically acceptable salt or prodrug thereof, is distributed as particles within the biocompatible polymer. In some embodiments, the codrug or a pharmaceutically acceptable salt or prodrug thereof, is in a mixture with the biocompatible polymer.

In certain embodiments, the device consists essentially of codrug or a pharmaceutically acceptable salt or prodrug thereof.

In some embodiments, the first constituent moiety is the same as the second constituent moiety. In other embodiments, the first constituent moiety is different from the second constituent moiety.

In certain embodiments, the first and second constituent moieties are directly linked through a covalent bond formed between a functional group of the first constituent moiety and a functional group of the second constituent moiety. In other embodiments, the first and second constituent moieties are linked to one another via a linking group that is covalently bonded to the first and second constituent moieties via functional groups thereon.

In preferred embodiments, the first constituent moiety is a corticosteroid. In preferred embodiments, the second constituent moiety is a corticosteroid, an antiproliferative compound, or an NSAID.

In preferred embodiments, the corticosteroid is selected from triamcinolone acetonide, fluocinolone acetate, fluocinolone acetonide, cortisone, hydrocortisone, and hydrocortisone ester.

In certain embodiments, the first constituent moiety is an antiproliferative agent and the second constituent moiety is an NSAID, with the proviso that the first constituent moiety is not floxuridine, and with the further proviso that when the first constituent moiety is 5-FU, the second constituent moiety is not flurbiprofen or indomethacin.

In preferred embodiments, the first constituent moiety is an antiproliferative agent and the second constituent moiety is a corticosteroid agent, with the proviso that when the antiproliferative agent is 5-FU, the corticosteroid is not fluocinolone acetonide, triamcinolone, triamcinolone acetonide, desoximetasone, or hydrocortisone-17-butyrate, and with the further proviso that the antiproliferative agent is not a 1-β-arabinofuranosylcytosine derivative.

Another aspect of the invention provides a method for treating a patient, comprising implanting or injecting into or otherwise administering to a patient a drug delivery device as described above, wherein the device is implanted in the prostate, cervix, bladder, bladder neck, anal submucosa, or the tissues surrounding the aforementioned tissues or organs.

Another aspect of the invention provides a method of inhibiting cell proliferation in a patient in need of treatment, comprising implanting or injecting into or otherwise administering to a patient a drug delivery device as described above, wherein the device includes a therapeutically effective amount of a codrug or a pharmaceutically acceptable salt or prodrug thereof.

In some embodiments, the method of inhibiting inflammation in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating fungal, viral and/or bacterial infection in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating neovascularization in a patient in need of treatment comprises implanting or injecting into or otherwise administering to a patient, a drug delivery device as described above, wherein the device includes a therapeutically effective amount of a codrug or a pharmaceutically acceptable salt or prodrug thereof.

In some embodiments, the method of treating localized immunomodulation in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating incontinence in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating inflammatory and/or neurogenic pain in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating tissue permeability changes in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating urinary retention in a patient in need of treatment comprises implanting or injecting into or otherwise administering to a patient a drug delivery device as described above, wherein the device includes a therapeutically effective amount of a codrug or a pharmaceutically acceptable salt or prodrug thereof.

In some embodiments, the method of treating hyperproliferation of tissue in a patient in need of treatment comprises implanting or injecting into or otherwise administering to a patient, a drug delivery device as described above, wherein the device includes a therapeutically effective amount of a codrug or a pharmaceutically acceptable salt or prodrug thereof.

In some embodiments, the method of treating muscle relaxation in a patient in need of treatment comprises implanting or injecting into or otherwise administering to a patient, a drug delivery device as described above, wherein the device includes a therapeutically effective amount of a codrug or a pharmaceutically acceptable salt or prodrug thereof.

In certain embodiments, the method further comprises implanting the device according to claim 1 in the prostate, cervix, bladder, bladder neck, anal submucosa, or the tissues surrounding the aforementioned tissues or organs.

In some embodiments, the treatment needed by the patient is for a genitourinary disorder. In certain embodiments, the genitourinary disorder is prostate cancer, prostatitis, cervical cancer, incontinence, a bladder disorder, benign prostatic hypertrophy (BPH), a chronic pelvic pain syndrome (e.g., irritable bowel syndrome, interstitial cystitis, prostatitis), uterine cancer, endometriosis, bladder cancer, sexual dysfunction (male and female), infertility, a sexually transmitted disease, or a urinary tract infection.

In some embodiments, the method further comprises implanting radioactive seeds. In certain embodiments, the method further comprises radiation therapy, chemotherapy, transurethral resection of the prostrate, transurethral microwave therapy, transurethral thermal therapy, or laser ablation.

In some embodiments, the device further comprises a stent, artificial sphincter, a penile protheses, a bulking agent, or a catheter. In certain embodiments, the stent may be a prostatic stent, urethral stent, etc.

A further aspect of the invention provides a kit comprising a drug delivery device as described above in association with instructions (written and/or pictorial) describing the use of the device for treatment or prevention of a genitourinary disorder and optionally, warnings of possible side effects and drug-drug interactions.

Still another aspect of the invention provides a method of manufacturing a drug delivery device, comprising providing a codrug comprising
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device is dimensioned to position two radiation seeds a predetermined distance apart.

In certain embodiments, the polymer matrix is non-bioerodible, while in other embodiments it is bioerodible. Exemplary non-bioerodible polymer matrices can be formed from polyhema, polyhydroxybutyrate (PHB), polyhydroxyvalerate (PHV), polycaprolactone, polyanhydrides, polyorthoesters, polyaminoacids (and “pseudo” polyaminoacids), polycyanoacrylates, polyphosphazenes, polyurethane, polysilicone, poly(ethylene-co-vinyl acetate), PVA, and derivatives and copolymers thereof.

Exemplary bioerodible polymer matrices can be formed from collagen, polyanhydride, polylactic acid, polyglycolic acid, polyorthoester, polyalkylcyanoacrylate, and derivatives and copolymers thereof.

In certain embodiments, the polymer matrix is chosen so as reduce interaction between the prodrug in the matrix and proteinaceous components in surrounding bathing fluid, e.g., by forming a matrix having physical (pore size, etc.) and/or chemical (ionized groups, hydrophobicity, etc.) characteristics which exclude proteins from the inner matrix, e.g., exclude proteins of greater than 100 kD, and even more preferably exclude proteins greater in size than 50 kD, 25 kD, 10 kD, or even 5 kD.

In certain embodiments, the polymer matrix is essentially non-release-rate-limiting with respect to the rate of release of a constituent moiety from the matrix.

In other embodiments, the subject polymer matrices influence the rate of release. For instance, the matrices can be derived to have charge or hydrophobicity characteristics which favor sequestration of the codrug over the constituent moieties. Likewise, the polymer matrix can influence the pH-dependency of the hydrolysis reaction, or create a microenvironment having a pH different than the bathing bodily fluid, such that hydrolysis, stability, and/or solubility of the prodrug or codrug is different within the matrix in comparison to the surrounding fluids. In such a manner, the polymer can influence the rate of release and the rate of hydrolysis of the prodrug or codrug, by differential electronic, hydrophobic or chemical interactions with the prodrug or codrug.

In many preferred embodiments, the duration of release from the polymer matrix of a therapeutically effective amount of a constituent moiety is at least 24 hours, and even more preferably may be at least 72 hours, 100, 250, 500 or even 750 hours. In certain embodiments, the duration of release of a constituent moiety from the polymer matrix is at least one week, more preferably two weeks, or even more preferably at least three weeks. In certain embodiments, the duration of release of a constituent moiety from the polymer matrix is at least one month, more preferably two months, and even more preferably six months.

In some embodiments, the codrug is selected from 5-FU covalently bonded to fluocinolone acetonide (FA) (I), 5-FU covalently bonded to naproxen (II), and 5-FU covalently bonded to diclofenac (III). Exemplary codrugs include:

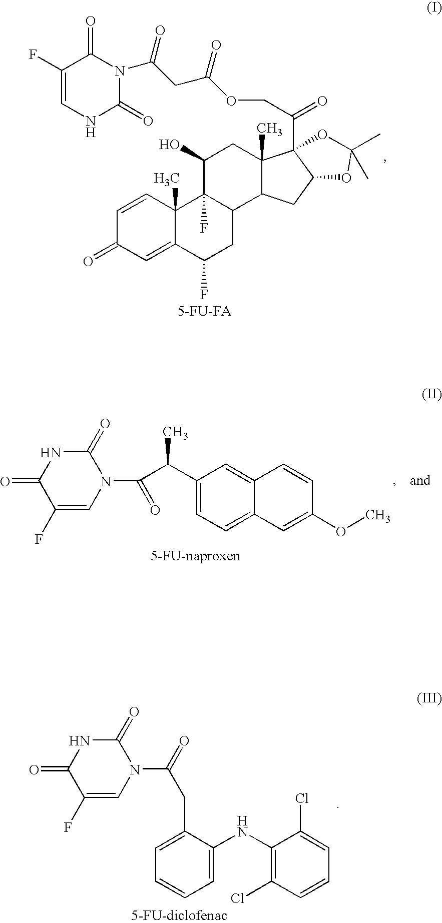

In certain embodiments, the codrug is present in the device in an amount between 1% and 100% by weight of the device, and even more preferably 5% to 50% by weight. Compositions including codrugs as discussed above may be used to treat a wide variety of genitourinary-related conditions, such as cell proliferation (e.g., cancer, restenosis, or other hyperproliferation), inflammation, pain (e.g., for analgesia or anesthesia), infection (e.g., a viral, bacterial, or fungal infection), infertility (including below normal fertility), impotency or compromised sexual function, abnormally high urinary urgency and/or frequency in a patient, incontinence, neovascularization, as well as for therapeutic immunomodulation and modulation of tissue permeability and/or muscle relaxation. In such embodiments, the device may be implanted, injected, or otherwise administered in the prostate, cervix, bladder, bladder neck, anal submucosa, or the tissues surrounding the aforementioned tissues or organs.

Exemplary genitourinary disorders that may be treated by the compositions and methods disclosed herein include prostate cancer, prostatitis, cervical cancer, incontinence, a bladder disorder, benign prostatic hypertrophy (BPH), a chronic pelvic pain syndrome (e.g., irritable bowel syndrome, interstitial cystitis, prostatitis), uterine cancer, endometriosis, bladder cancer, sexual dysfunction (male and female), infertility, sexually transmitted diseases, and urinary tract infections. Treatment regimens may include radiation therapy, chemotherapy, transurethral resection of the prostrate, transurethral microwave therapy, transurethral thermal therapy, and/or laser ablation in addition to the subject compositions.

In another aspect, the invention provides a kit comprising a drug delivery device as described herein in association with instructions (written and/or pictorial) describing the use of the device for treatment or prevention of a genitourinary disorder and, optionally, warnings of possible side effects and drug-drug interactions.

### BRIEF DESCRIPTION OF THE DRAWINGS

FIG. 1 is a time-dependent graph of remaining 5-FU-FA codrug in a codrug device according to the present invention from a dog prostate study.

FIG. 2 is a graph of the amount (mean) of 5-FU-FA codrug remaining from a codrug device according to the present invention from a rabbit liver study.

### DETAILED DESCRIPTION OF THE INVENTION

I. Overview

Combining radiation and 5-FU to augment effects on prostate cancer cell death is one aspect of the present invention. Without being limited to a particular theory, it is also possible that radiation is a chemosensitizer of 5-FU.

The combination of both naproxen and 5-FU as a codrug was tested in canine prostate in combination with full-dose brachytherapy with no adverse effects. A similar combination was used in a tissue healing study in rabbits with no adverse effects. The delivery technique was designed and has been used in drug delivery in the eye.

In view of the foregoing, an aspect of the present invention is a method of using drug delivery devices for the treatment of genitourinary diseases and disorders including, but not limited to, BPH, prostate cancer, prostatitis, including chronic prostatitis, cervical cancer, bladder cancers, and cancers of the urethra.

Another aspect of the present invention is a method for treating prostate diseases and disorders using implantable, injectable or otherwise administrable drug delivery devices which do not need to be frequently re-administered or repeated, both alone and in combination with radiation treatments, including brachytherapy and other indicated radiation therapies.

Another aspect of the present invention is the delivery of effective amounts of therapeutic agents, including codrugs. Codrugs are described in U.S. Pat. No. 6,051,576 to Ashton, et al., the entirety of which is incorporated by reference herein.

Another aspect of the present invention is a codrug of one or more pharmacologically active compounds in the following classes of agents: anticancer or antiproliferative agents, including but not limited to 5-FU, adriamycin and related compounds; anti-inflammatory and analgesic agents including but not limited to, naproxen; NSAIDs, including but not limited to, flurbiprofen and indomethacin; antibiotic agents, including but not limited to, amikacin, tobramycin, and quinolones; antiandrogens, including but not limited to, LHRH agonists or progestational agents; alpha-blockers, including but not limited to, analogs of phenoxybenzamine and prazosin; and corticosteroids, including but not limited to dexamethasone and triamcinolone acetonide. Therefore, codrugs of the present invention may include one or more drugs combined as explained in the above-mentioned patent to Ashton, et al., and below. Furthermore, codrugs of the present invention also include codrugs of a single compound (e.g., a codrug in which the two constituent drugs are the same agent). Those of skill in the art will readily appreciate that the present invention is not limited to the specific agents listed above, but extends to compounds with similar therapeutic effects and for which the use is indicated for the particular disease state of interest. More detailed lists of the therapeutic agents to which the present invention extends can be found in, e.g., Goodman & Gilman's The Pharmacological Basis of Therapeutics (110th ed., The McGraw-Hill Companies, Inc., 2001), Remington's Pharmaceutical Sciences (18th ed., Mack Publishing Co., 1990), The Merck Index (12th ed., Merck Research Laboratories, 1996), and other such volumes.

The linker L may be either a direct bond between individual constituent moieties, or it may include a linking group. The first and second constituent moieties of the codrugs of the present invention may be linked via reversible covalent bonds such as ester, amide, carbamate, carbonate, cyclic ketal, thioester, thioamide, thiocarbamate, thiocarbonate, xanthate and phosphate ester bonds, so that, at the required site in the body, they are cleaved to regenerate the active forms of the constituent pharmaceutically active agents.

The covalent bonds between residues include a bonding structure such as: 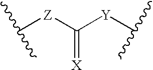
wherein Z is O, N, —CH₂—, —CH₂—O—, or —CH₂—S—, Y is O or N, and X is O or S. The rate of cleavage of the individual moieties can be controlled by the type of bond, the choice of constituent moieties, and the physical form of the codrug. The lability of the selected bond type may be enzyme-specific. In some embodiments according to the present invention, the bond is selectively labile in the presence of an esterase. In other embodiments of the invention, the bond is chemically labile, e.g., to acid- or base-catalyzed hydrolysis.

In preferred embodiments according to the present invention, the linking group L does not include a sugar, a reduced sugar, a pyrophosphate, or a phosphate group.

The physiologically labile linkage may be any linkage that is labile under conditions approximating those found in physiologic fluids. The linkage may be a direct bond (for instance, ester, amide, carbamate, carbonate, cyclic ketal, thioester, thioamide, thiocarbamate, thiocarbonate, xanthate, phosphate ester, sulfonate, or a sulfamate linkage) or may be a linking group (for instance a C₁-C₁₂ dialcohol, a C₁-C₁₂ hydroxyalkanoic acid, a C₁-C₁₂ hydroxyalkylamine, a C₁-C₁₂ diacid, a C₁-C₁₂ aminoacid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the codrug has not been solubilized in a physiologic fluid. Thus, while some codrugs according to the present invention may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g., non-aqueous solvents such as acetone). Thus, the labile linkages are such that, when the codrug is dissolved in an aqueous solution, the reaction is driven to the hydrolysis products, which include the constituent moieties set forth above.

Codrugs for preparation of a drug delivery device according to the present invention may be synthesized in the manner illustrated in one of the synthetic schemes below. In general, where the first and second pharmaceutically active moieties are to be directly linked, the first moiety is condensed with the second moiety under conditions suitable for forming a linkage that is labile under physiologic conditions. In some cases it is necessary to block some reactive groups on one, the other, or both of the moieties. Where the pharmaceutically active moieties are to be covalently linked via a linker, such as oxamethylene, succinic acid, or diglycolic acid, it is advantageous to first condense the first pharmaceutically active moiety with the linker. In some cases it is advantageous to perform the reaction in a suitable solvent, such as acetonitrile, in the presence of suitable catalysts, such as carbodiimides including EDCI (1-ethyl-3-(3-dimethylaminopropyl)-carbodiimide) and DCC (DCC: dicyclohexylcarbodiimide), or under conditions suitable to drive off water of condensation or other reaction products (e.g., reflux), or a combination of two or more thereof. After the first pharmaceutically active moiety is condensed with the linker, the combined first moiety and linker may then be condensed with the second pharmaceutically active moiety. Again, in some cases it is advantageous to perform the reaction in a suitable solvent, such as acetonitrile, in the presence of suitable catalysts, such as carbodiimides including EDCI and DCC, or under conditions suitable to drive off water of condensation or other reaction products (e.g., reflux), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

A person of skill in the art will recognize that, while diacids, dialcohols, amino acids, etc., are described as being suitable linkers, other linkers are contemplated as being within the present invention. For instance, while the hydrolysis product of a codrug according to the present invention may comprise a diacid, the actual reagent used to make the linkage may be, for example, an acylhalide such as succinyl chloride. A person of skill in the art will recognize that other possible acid, alcohol, amino, sulfato, and sulfamoyl derivatives may be used as reagents to make the corresponding linkage.

Where the first and second pharmaceutically active moieties are to be directly linked via a covalent bond, essentially the same process is conducted, except that in this case there is no need for a step of adding a linker. The first pharmaceutically active moiety and second pharmaceutically active moieties are merely combined under conditions suitable for forming the covalent bond. In some cases it may be desirable to block certain active groups on one, the other, or both of the pharmaceutically active moieties. In some cases it may be desirable to use a suitable solvent, such as acetonitrile, a catalyst suitable to form the direct bond, such as carbodiimides including EDCI and DCC, or conditions designed to drive off water of condensation (e.g., reflux) or other reaction by-products.

A person of skill in the art will recognize that, while in most cases the first and second moieties may be directly linked in their original form, it is possible for the active groups to be derivatized to increase their reactivity. For instance, where the first moiety is an acid and the second moiety is an alcohol (i.e., has a free hydroxyl group), the first moiety may be derivatized to form the corresponding acid halide, such as an acid chloride or an acid bromide. A person of skill in the art will recognize that other possibilities exist for increasing yield, lowering production costs, improving purity, etc., of the codrug according to the present invention by using conventionally derivatized starting materials to make codrugs according to the present invention.

Exemplary reaction schemes according to the present invention are illustrated in Schemes 1-4, below. These Schemes can be generalized by substituting other therapeutic agents having at least one functional group that can form a covalent bond to another therapeutic agent having a similar or different functional group, either directly or indirectly through a pharmaceutically acceptable linker. A person of skill in the art will appreciate that these schemes also may be generalized by using other appropriate linkers. 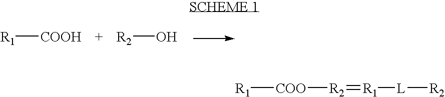
wherein L is an ester linker —COO—, and R₁ and R₂ are the residues of the first and second constituent moieties or pharmacological moieties, respectively. 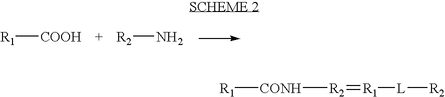
wherein L is the amide linker —CONH—, and R₁ and R₂ have the meanings given above. 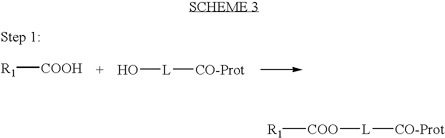
wherein Prot is a suitable reversible protecting group.

Step 2: R₁—COO-L-CO-Prot→R₁—COO-L-COOH

Step 3: R₁—COO-L-COOH+R₂—OH→R₁—COO-L-COOR₂
wherein R₁, L, and R₂ have the meanings set forth above. 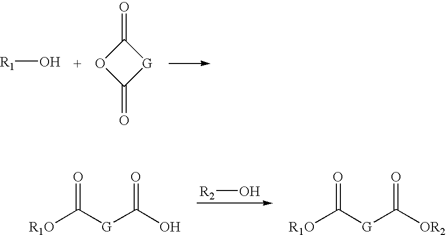
wherein R₁ and R₂ have the meanings set forth above and G is a direct bond, an C₁-C₄ alkylene, a C₂-C₄ alkenylene, a C₂-C₄ alkynylene, or a 1,2-fused ring, and G together with the anhydride group completes a cyclic anhydride. Suitable anhydrides include succinic anhydride, glutaric anhydride, maleic anhydride, diglycolic anhydride, and phthalic anhydride.

Another aspect of the present invention is the use of implantable drug delivery devices to delivery therapeutic agents to a localized tissue volume or mass. While numerous drug delivery devices are usable in the present invention, one aspect of the present invention is the utilization of the structures of the devices described in U.S. Pat. Nos. 5,378,475, 5,902,598, 5,836,935, 5,773,019, and 6,375,972, and relatively simple drug (powder) pellets. When a method in accordance with the present invention necessitates the use of more than one such device, another aspect of the present invention is utilizing two or more different devices as the drug delivery device.

Another aspect of the present invention provides a method of treating prostate cancer. In some embodiments, drug delivery devices are implanted into the prostate, or into the tissues immediately adjacent to the prostate. The drug delivery devices, detailed below, release a therapeutic agent or drug over time to treat the prostate cancer, or symptoms associated with the prostate cancer, or symptoms associated with other treatment modalities for the prostate cancer. According to one aspect of the invention, the drug delivery devices are embodied as spacers between brachytherapy seeds. The therapeutic spacers are loaded with, or are composed at least in part of, a therapeutic agent which includes, but is not limited to, a codrug of 5-FU/triamcinolone acetonide.

As described above, brachytherapy treatments generate pain, edema, and associated voiding problems. In some embodiments, the codrug selected for release from a brachytherapy drug delivery spacer decreases, and preferably eliminates, pain, swelling, and/or voiding symptoms following brachytherapy, and may also enhance (or be enhanced by) radiation therapy. As well established and understood by those of skill in the art, and as described herein, known therapeutic compounds, such as 5-FU and triamcinolone acetonide, have beneficial effects in the treatment of these symptoms, especially in conjunction with brachytherapy.

Therapeutic agent delivering spacers may be placed between the radioactive seeds in the delivery needles of the brachytherapy machine, to keep the radioactive seeds in their proper predetermined positions. The simultaneous use of antiinflammatory agents that have a controlled and prolonged release rate also limits the brachytherapy side effects, while the localized delivery of the agents to the prostate does not impose the chemotherapeutic load on the patient's entire system that is a significant shortcoming of some prior systemic administration protocols.

More specifically, some embodiments of a therapeutic spacer in accordance with the present invention releases a combination of two classes of drugs that have been used extensively in prostate cancer treatment protocols. One traditional regimen has been intravenous 5-FU with oral corticosteroids. As already demonstrated, the combination of the two drug types has been used together in tumor models without deleterious effects.

As described in the aforementioned '576 patent, the codrugs rely on a labile bond between the two drugs that makes them poorly soluble in water. This codrug may be a powder that, when compressed into a pellet, or incorporated into a drug core or reservoir as described in the aforementioned drug delivery device patents, has a sustained release when placed in tissue, such as the prostate. An aqueous environment in vivo causes the codrug to hydrolyze and release active drugs from the pellet or out of the device core, because the labile bond quickly breaks in an aqueous environment, releasing the regenerated parent drugs (see, e.g., FIG. 1 of the '576 patent and associated text). A triamcinolone acetonide/5-FU codrug therapeutic spacer has been shown to have a half-life in the prostate of 20 days. The amount of drug implanted can be dramatically smaller than the normal systemic dose given to achieve similar tissue levels. The amount released locally is constant so that the local levels are effective, but the systemic levels are negligible. This release rate in tissue is similar to that in buffered solution. The codrug combination of triamcinolone acetonide/5-FU has also been tested extensively in treatment of proliferative eye disease in rabbit models. The combination was not only very successful, but there were no drug-related toxic effects clinically or in histopathology.

A canine study was performed where preplanned brachytherapy was given using ¹⁰³Pd and the codrug naproxen/5-FU. Once again, no side effects or toxicity were observed clinically or histologically. Alternatively, triamcinolone acetonide/5-FU codrug can be chosen over the naproxen/5-FU codrug because of the greater potency of corticosteroids. Plus, corticosteroids have been used more extensively in prostate cancer than NSAIDs and been shown to decrease post-brachytherapy edema when given systemically.

Another aspect of the present invention is the treatment of prostatitis and/or an infected prostate gland using a codrug or codrugs of linked antibiotic agents, anti-inflammatory agents, including NSAIDs, and combinations and permutations thereof, as at least one of the therapeutic agents in a drug delivery device placed in the prostate.

There are numerous aspects of the present invention which extend to the delivery of therapeutically effective amounts of therapeutic agents to numerous distinct anatomical sites, both alone and in conjunction with radiotherapy. One aspect of the present invention is the implantation of a drug delivery device which releases a therapeutically effective amount of one or more of the agents described herein, wherein the device is not a brachytherapy spacer, but is implanted in the prostate, cervix, bladder, or other diseased portion of the genitourinary system, and enhances, or is enhanced by, radiotherapy. Such radiotherapy includes, as will be readily appreciated from the descriptions herein, localized radiotherapy, such as brachytherapy seed implantation or radioactive rod, needle, or wire implantation.

Another aspect of the present invention is the utilization of prodrugs in the treatment of disease states described herein. Prodrugs are described in, for example, U.S. Pat. No. 5,681,964. Thus, methods of the present invention also include using a drug delivery device, as described herein, which is loaded with a prodrug. The therapeutic agent of the prodrug is selected to treat the disease state of interest, also as described herein. More than one prodrug can be loaded into the device, or more than one device can be used (with one prodrug per device), or combinations thereof, and the agents delivered, so that the treatment of the disease state can benefit from the synergisms of the combined use of the therapeutic agents described herein. This method can be combined with radiotherapy treatments, such as brachytherapy, as also described herein.

Another aspect of the present invention is the implantation or injection into the prostate of a drug delivery device to treat chronic prostatitis. In some embodiments, the drug delivery device releases one or more therapeutic agents to the prostate; where prostate cancer is not being treated, brachytherapy may not be necessary. The one or more therapeutic agents delivered to the prostate may be selected from among those agents known to have beneficial therapeutic effects in treating chronic prostatitis, both sterile and infectious, as described herein and as will be readily apparent to those of skill in the art.

Another aspect of the present invention includes selecting the mode of delivery of the therapeutic agent based at least in part on the solubility of the agent in vivo. When the agent of interest is highly soluble, it is preferable, although not necessary, that the agent be delivered in codrug form, so that it is made bioavailable only when it is released into the target tissue. Thus, by effectively rendering the agents less hydrophilic by forming codrugs of the agent of interest, the agent is more effectively delivered to the target tissues according to the present invention.

The brachytherapy spacer(s) may be either the standard, non-therapeutic agent delivering spacer(s), or one of the drug delivery device spacers including the specific therapeutic agent or combinations of agents, including codrugs of agents. The needles will then deploy the ¹⁰³Pd and spacer. Once all the needles are removed, a volume study will be performed. Cystoscopy will be performed to check for intravesical ¹⁰³Pd seeds and the Foley will be replaced. Oral pain medications will be used based on tolerances, sensitivities, and allergies, and interactions.

II. Definitions

The term “active” as used herein means therapeutically or pharmacologically active.

The term “ED₅₀” means the dose of a biologically active moiety that produces 50% of its maximum response or effect.

The term “IC₅₀” means the dose of a biologically active moiety that inhibits a biological activity by 50%.

The term “LD₅₀” means the dose of a biologically active moiety that is lethal in 50% of test subjects.

The term “therapeutic index” refers to the therapeutic index of a biologically active moiety defined as LD₅₀/ED₅₀.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or codrugs thereof, prior to conjugation.

As used herein, the term “constituent moiety” means one of two or more biologically active moieties so linked as to form a codrug according to the present invention as described herein. In some embodiments according to the present invention, two molecules of the same constituent moiety are combined to form a dimer. In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or codrugs thereof, prior to conjugation.

“Log P” refers to the logarithm of P (Partition Coefficient). P is a measure of how well a substance partitions between octanol and water. P itself is a constant for a given molecule. It is defined as the ratio of concentration of compound in aqueous phase to the concentration of compound in an immiscible solvent, as the neutral molecule.
Partition Coefficient, P=[Organic]/[Aqueous] where [ ]=concentration
Log P=log₁₀ (Partition Coefficient)=logo₁₀ P

In practice, the Log P value will vary according to the conditions under which it is measured. A Log P value of 1 means that the concentration of the compound is ten times greater in the organic phase than in the aqueous phase. The increase in a log P value of 1 indicates a ten-fold increase in the concentration of the compound in the organic phase as compared to the aqueous phase. Compounds with log P values greater than 5 are considered as having very low aqueous solubility. In general, compounds having log P values between 7 and 10 are considered almost insoluble in aqueous media.

In the context of referring to the codrug according to the present invention, the term “residue of a constituent moiety” means that part of a codrug that is structurally derived from a constituent moiety apart from the functional group through which the moiety is linked to another constituent moiety. For instance, where the functional group is —NH₂, and the constituent group forms an amide (—NH—CO—) bond with another constituent moiety, the residue of the constituent moiety is that part of the constituent moiety that includes the —NH— of the amide, but excluding the hydrogen (H) that is lost when the amide bond is formed. In this sense, the term “residue” as used herein is analogous to the sense of the word “residue” as used in peptide and protein chemistry to refer to a residue of an amino acid in a peptide.

The terms “drug” and “pharmaceutical” are interchangeable as used herein and have their art-recognized meanings.

As used herein, the phrase “the codrug is relatively lipophilic,” means that the codrug is more lipophilic than one or more of the constituent moieties that it comprises. In some embodiments according to the present invention, the codrug is more lipophilic than only one of the constituent moieties. In other embodiments according to the present invention, the codrug is more lipophilic than more than one of the constituent moieties, and in particular embodiments according to the present invention, the codrug is more lipophilic than all the constituent moieties of the codrug.

A “patient” or “subject” to be treated by the subject method can mean either a human or non-human animal.

A “pharmacological moiety” is a moiety that, when active or when activated, can cause an intended medical effect. Pharmacological moieties typically cause these effects when made to interact with a drug target (generally in the body of a subject to which the moiety has been administered, particularly a human or mammal that is a model of a human disease or condition, but possibly also in an animal, such as a bird or mammal, in a veterinary administration of the moiety).

The phrase “pharmaceutically acceptable carrier” as used herein means a pharmaceutically acceptable material, composition or vehicle, such as a liquid or solid filter, diluent, excipient, solvent or encapsulating material, involved in carrying or transporting the subject regulators from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) ethyl alcohol; (19) phosphate buffer solutions; and (20) other non-toxic compatible substances employed in pharmaceutical formulations.

“Pharmaceutically acceptable salt” refers to a cationic salt formed at any acidic (e.g., hydroxamic or carboxylic acid) group, or an anionic salt formed at any basic (e.g., amino or guanidino) group. Such salts are well known in the art. See e.g., PCT Publication 87/05297, incorporated herein by reference. Such salts are made by methods known to one of ordinary skill in the art. It is recognized that the skilled artisan may prefer one salt over another for improved solubility, stability, formulation ease, price and the like. Determination and optimization of such salts is within the purview of the skilled artisan's practice. Preferred anions include halides (such as chloride), sulfonates, carboxylates, phosphates, therapeutically active carboxylates, and the like.

“Physiological conditions” describe the conditions inside an organism, i.e., in vivo. Physiological conditions include the acidic and basic environments of body cavities and organs, enzymatic cleavage, metabolism, and other biological processes, and preferably refer to physiological conditions in a vertebrate, such as a mammal.

A “prodrug” is a moiety that is generally not pharmacologically active. However, when activated, typically in vivo by enzymatic or hydrolytic cleavage to convert the prodrug to an active biological moiety, the administration of the prodrug to the individual will have had the intended medical effect. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

The phrases “systemic administration,” “administered systemically,” “peripheral administration”, and “administered peripherally” as used herein mean the administration of a biologically active moiety, codrug, or other material other than directly into the central nervous system, such that it enters the patient's system and, thus, is subject to metabolism and other like processes, for example, subcutaneous administration.

The term “treatment” is intended to encompass also prophylaxis, therapy and cure. The patient receiving this treatment is any animal in need, including primates, particularly humans, and other mammals such as equines, cattle, swine and sheep; and poultry and pets in general.

A “substitution” or “substituent” on a small organic molecule generally refers to a valency on a multivalent atom occupied by a moiety other than hydrogen, e.g., a position on a chain or ring exclusive of the member atoms of the chain or ring. Such moieties include those defined herein and others as known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulfhydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

The terms “amine” and “amino” are art-recognized and refer to both unsubstituted and substituted amines as well as ammonium salts, e.g., as can be represented by the general formula: 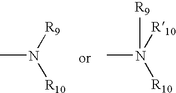
wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term “alkylamine” as used herein means an amine group, as defined above, having at least one substituted or unsubstituted alkyl attached thereto. Amino groups that are positively charged (e.g., R′₁₀ is present) are referred to as “ammonium” groups. In amino groups other than ammonium groups, the amine is preferably basic, e.g., its conjugate acid has a pKₐ above 7.

The terms “amido” and “amide” are art-recognized as an amino-substituted carbonyl, such as a moiety that can be represented by the general formula: 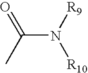
wherein R₉ and R₁₀ are as defined above. In certain embodiments, the amide will include imides. In general, when the oxygen of the above formula is replace by sulfur, the formula represents a “thioamide”.

The term “carbonyl” is art-recognized and includes such moieties as can be represented by the general formula: 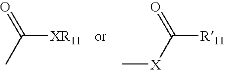
wherein X is a bond or represents an oxygen or a sulfur, and R₁₁ represents a hydrogen, hydrocarbon substituent, or a pharmaceutically acceptable salt, R₁₁ represents a hydrogen or hydrocarbon substituent. Where X is an oxygen and R₁₁ or R₁₁ is not hydrogen, the formula represents an “ester”. Where X is an oxygen, and R₁₁, is as defined above, the moiety is referred to herein as a carboxyl group, and particularly when R₁₁, is a hydrogen, the formula represents a “carboxylic acid”. Where X is an oxygen, and R₁₁ is hydrogen, the formula represents a “formate”. In general, where the oxygen atom of the above formula is replaced by sulfur, the formula represents a “thiocarbonyl” group. Where X is a sulfur and R₁₁ or R₁₁ is not hydrogen, the formula represents a “thioester”. Where X is a sulfur and R₁₁, is hydrogen, the formula represents a “thiocarboxylic acid”. Where X is a sulfur and R₁₁, is hydrogen, the formula represents a “thioformate”. On the other hand, where X is a bond, R₁₁ is not hydrogen, and the carbonyl is bound to a hydrocarbon, the above formula represents a “ketone” group. Where X is a bond, R₁₁ is hydrogen, and the carbonyl is bound to a hydrocarbon, the above formula represents an “aldehyde” or “formyl” group.

“Carbamate” refers to the group having the following general structure 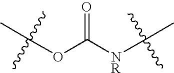
wherein R represents hydrogen or a hydrocarbon substituent.

A “thiocarbamate” refers to a variant of the above group wherein the oxygen of the carbonyl is replaced by sulfur.

“Carbonate” refers to the group having the following general structure of

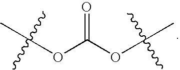

A “thiocarbonate” refers to a variant of the above structure wherein the oxygen of the carbonyl is replaced by sulfur.

“Cyclic ketal” refers to a cyclic aliphatic group including two oxygen atoms, such as moieties having one of the following general structures: 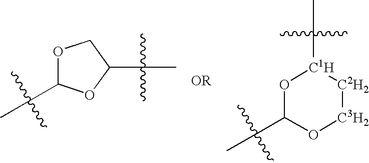
wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

“Phosphate ester” has refers to a group having the following general structure 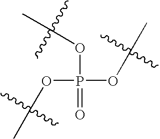
wherein each of the groups attached to the oxygens may be hydrogen, hydrocarbon, or a counterion (such as sodium) or other substituents as defined above.

A cyclic phosphate ester has the following general structure 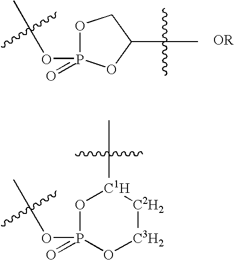
wherein substituents, such as the one depicted on C¹, could also, alternatively or additionally, be present at any other position(s) on the ring, such as on C₂ or C³, and/or two substituents can be present on the same position of the ring. Two carbons of the three carbons, C¹, C², and C³, together may be included in another ring structure having from 4 to 8 atoms in the ring structure.

“Guanidino” refers to a group having the following general structure 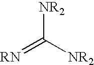
wherein each R may be, independently for each occurrence, a hydrogen or a hydrocarbon substituent. Two R's taken together may form a ring. The general structure may thus be part of one ring or a polycyclic structure.

“Amidines” are represented by the general formula 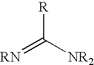
and are basic groups wherein each R may be, independently for each occurrence, a hydrogen or a hydrocarbon substituent. Two R taken together may form a ring.

“Hydrocarbon substituents” are moieties that include at least one C—H bond, and include groups such as alkyl, heteroalkyl, aryl, heteroaryl, carbocyclic aliphatic, and heterocyclic aliphatic groups.

“Heteroatom” refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

“Heterocyclic aliphatic ring” is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

“Heteroalkyl” is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono- and di- C1-C3 alkylamino, methylphenylamino, methylbenzylamino, C1-C3 alkylamido, carbamamido, ureido, guanidino).

A “xanthate” refers to the group having the following general structure 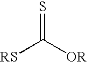
wherein R represents a hydrocarbon substituent.

III. Exemplary Codrugs

In some embodiments, the codrugs of the invention are formed by covalent conjugation of two or more constituent moieties. The constituent moieties can be linked to form a single codrug by reversible covalent bonds such that, at the desired site in the body, the covalently-linked constituent moieties are cleaved to regenerate the active forms of the constituent moieties, or the prodrug precursors to the biologically active moieties of interest. The rate of cleavage of the constituent moieties can be controlled by the type of the bond linking the constituent moieties, the choice of constituent moieties, and the physical form of the moieties. Codrugs according to the present invention are labile in water, serum, or other bodily fluids, and regenerate the biologically active moieties or prodrugs thereof. In some embodiments, the codrugs of the present invention have very low solubility in one or more of serum and other bodily fluids, and are quickly hydrolyzed to regenerate the biologically active moieties or prodrugs thereof upon dissolution in a biological environment.

Each constituent moiety possesses one or more functional groups that are capable of forming a labile bond with another constituent moiety, or with a linkage that is linked to a constituent moiety. Suitable labile bonds include ester, amide, carbamate, carbonate, cyclic ketal, thioester, thioamide, thiocarbamate, thiocarbonate, xanthate, phosphate ester, sulfonate, or a sulfamate, anhydride, urea, guanidino, and sulfonamido bonds. Suitable functional groups for forming these bonds include amino, carboxylic acid, hydroxy, thiol, and sulfonate groups. Suitable linking groups include diacids, diamines, amino acids, hydroxy acids, hydroxy amines, dialcohols, etc.

The constituent moieties may be any biologically active moieties that possess one or more functional groups that may form hydrolyzable bonds with themselves (e.g., dimers, trimers, etc.), other biologically active moieties, or with a linkage if one is used. The constituent moieties may be, for instance, analgesic compounds such as lidocaine, benzodiazepam, tramadol, and related compounds; anti-inflammatory steroidal compounds (corticosteroids); NSAIDs such as diclofenac, naproxen, ketorolac, flurbiprofen, and indomethacin; antibiotic compounds; anti-fungal compounds such as fluconazole and related compounds; antiviral compounds such as foscarnet sodium, trifluorothymidine, acyclovir, ganciclovir, dideoxyinosine (ddI), dideoxycytidine (ddC); antiproliferative compounds such as 5-FU, adriamycin and related compounds; immunomodulatory compounds such as muramyl dipeptide and related compounds; cell transport/mobility impeding agents such as colchicine, vincristine, cytochalsian B, and related compounds; cytokines and peptides/proteins such as cyclosporin, insulin, growth factor or growth hormones; etc.

Antiproliferative agents that are suitable for R₁ possess one or more functional groups that may react with either a functional group on R₂ or a linkage to form a bond. Exemplary functional groups possessed by R₁ include hydroxy groups, amine groups, carboxylate groups (including carboxylic acids and esters), acid anhydride groups, thiol groups, sulfonyl halide groups, etc. Preferred functional groups are —OH, —NH₂, —CO₂H, and —CO₂— groups (where the dash indicates bonding to the residue of the antiproliferative compound).

Exemplary antiproliferative agents include anthracyclines, vincaalkaloids, purine analogs, pyrimidine analogs, inhibitors of pyrimidine biosynthesis, and/or alkylating agents. Antiproliferative compounds suitable as one or more constituent moieties in the present invention include: adriamycin, alitretinoin (9-cis-retinoic acid); amifostine; arabinosyl 5-azacytosine; arabinosyl cytosine; 5-aza-2′-deoxycytidine; 6-azacytidine; 6-azauridine; azaribine; 6-azacytidine; 5-aza-2′-deoxycytidine; bexarotene (4-[1-(5,6,7,8-tetrahydro-3,5,5,8,8-pentamethyl-2-naphthalenyl)ethenyl]benzoic acid); bleomycin; capecitabine (5′-deoxy-5-fluoro-cytidine); chlorambucil; cladribine; cytarabine; cyclocytidine; daunorubicin; 3-deazauridine; 2′-deoxy-5-fluorouridine; 5′-deoxy-5-fluorouridine; docetaxel; doxorubicin; epirubicin; estramustine; etoposide; exemestane (6-methylenandrosta-1,4-diene-3,17-dione); fludarabine; fludarabin phosphate; fluorocytosine; 5-fluorouracil (5-FU); 5-fluorouridine; 5-fluoro-2′-deoxyuridine (FUDR); gemcitabine; hydroxyurea; idarubicin; irinotecan; melphalan; methotrexate; 6-mercaptopurine; mitoxantrone; paclitaxel; pentostatin; N-phosphonoacetyl-L-aspartic acid; prednimustine; pyrazofurin; streptozocin; temozolomide; teniposide; 6-thioguanine; tomudex; topotecan; 5-trifluoromethyl-2′-deoxyuridine; valrubicin (N-trifluoroacetyladriamycin-14-valerate); vinorelbine; other modified nucleotides and nucleosides, or a pharmaceutically acceptable salt thereof. Preferred antiproliferative agents are paclitaxel, docetaxel, methotrexate, and 5-FU. Each of these antiproliferative compounds possesses one or more functional groups as defined above, and all are thus capable of being linked to one or more of the same antiproliferative compound, a different antiproliferative compound, or a different pharmaceutically active compound, having a similar or different functional group, either directly or indirectly through a pharmaceutically acceptable linker.

Exemplary moieties that may be useful in a codrug for treating sexual dysfunction include phosphodiesterase-5 inhibitors (such as sildenafil, cialis, and vardenafil), angiontensin receptor blockers (such as lotarsan, candesartan, telmisartan, and valsartan), α-adrenergic blockers (such as atipamezole and phentolamine), apomorphine, minoxidil, yohimbine, pentoxyfylline, phentolamine, general phosphodiesterase inhibitors (such as papaverine), alyrosal, nitric oxide synthetase substrates (such as arginine), vasodilators (such as vasodilan and phentolamine), trazodone, prostaglandin E1 (or an analog or a derivative thereof, such as alprostadil; see, for example, U.S. Pat. No. 6,486,207), phosphodiesterase inhibitors, melanocortin receptor agonists (see, for example, U.S. Pat. Nos. 6,376,509 and 6,534,503), and nitric oxide donors (such as organic nitrites, nitrosoureas, nitrosocarbamates, and nitrosoamides).

Steroids, such as steroid hormones, are also contemplated for use as one or more constituent moieties according to the invention. Suitable steroids include estrogens and androgens, progesterone, estradiol, testosterone, dihydrotestosterone, androstenediol, androstenedione, dehydroepiandrosterone (DHEA), DHEA-S, estriol, estrone, pregnenolone, 17 a-hydroxypregnenolone, and 17-hydroxyprogesterone. A pharmaceutically acceptable analog, prodrug, or derivative of these hormones, such as methyl testosterone, dehydroepiandrostenedione, can be used in place of the naturally occurring hormones.

Suitable corticosteroids for use as one or more constituent moieties according to the present invention include: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide. Each of these corticosteroid moieties possesses one or more functional groups as defined above, and all are thus capable of being linked to one or more of the same corticosteroid, a different corticosteroid, or a different pharmaceutically active moiety.

Preferred corticosteroid moieties for preparing codrugs according to the present invention include moieties of the formula: 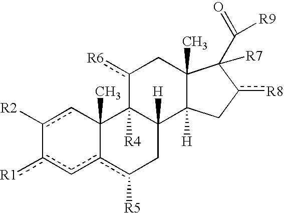
wherein R1 is ═O, —OH, or —(CH₂)₁₋₄Cl;
R2 is H, C₁₋₄alkyl, Cl, or Br;
R4 is H, F, or Cl;
R5 is H, F, Cl, CH₃, or —CHO;
R6 is H, OH, or Cl;
R7 is H, OH, CH₃, O—COCH₃, O(CO)OCH₂CH₃, O—(CO)-2-furanyl, or O—C(O)—(CH₂)₂CH₃;
R8 is H, CH₃, OH, ═CH₂, or together R7 and R8 form, together with the adjacent carbon atoms to which they are attached: 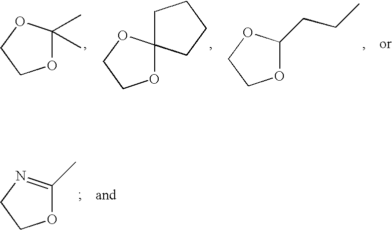
R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and
wherein the bonds indicated by [symbol] are either double or single bonds.

One skilled in the art will recognize that the class of corticosteroid compounds is a distinct class of steroids that does not include estrogens or androgens.

Illustrative examples of suitable β-lactam antibiotics include, amoxicillin, ampicillin, amylpenicillin, apalcillin, azidocillin, azlocillin, aztreonam, bacampicillin, benzylpenicillinic acid, biapenem, cefaclor, cefadroxil, cefamandole, cefatrizine, cefazedone, cefazolin, cefbuperazone, cefcapene pivoxil, cefclidin, cefdinir, cefditoren, cefepime, cefetamet, cefixime, cefinenoxime, cefinetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsolodin, ceftazidime, cefteram, ceftezole, ceftibuten, ceftiofur, ceftizoxime, ceftriaxone, cefuroxime, cefuzonam, cephacetrilic acid, cephalexin, cephaloglycin, cephaloridine, cephalosporin C, cephalothin, cephamycins, cephapirinic acid, cephradine, clometocillin, cloxacillin, cyclacillin, dicloxacillin, fenbenicillin, flomoxef, floxacillin, hetacillin, imipenem, lenampicillin, loracarbef, meropenem, metampicillin, moxalactam, norcardicins (e.g., norcardicin A), oxacillin, panipenem, penicillin G, penicillin N, penicillin O, penicillin S, penicillin V, phenethicillin, piperacillin, pivampicillin, pivcefalexin, propicillin, sulbenicillin, sultamicillin, talampicillin, temocillin, ticarcillin, and tigemonam. Each of the above-identified β-lactam antibiotics possesses at least one functional group capable of forming a covalent bond to at least one other pharmaceutically effective moiety having at least one functional group, either directly or via a labile linker.

Antibiotic compounds suitable as one of more constituent moieties in the present invention include: metronidazole, ciprofloxacin, amikacin, tobramycin, quinolones, etc.

NSAID compounds that are suitable for R₂ possess one or more functional groups that may react with either a functional group on R₁ or a linkage to form a bond. Exemplary functional groups possessed by R₂ include hydroxy groups, amine groups, carboxylate groups (including carboxylic acids and esters), acid anhydride groups, thiol groups, sulfonyl halide groups, etc. Preferred functional groups are —OH, —NH₂, —CO₂H (including —CO₂—) groups, (the dashes indicating bonding to the residue of the antiproliferative compound).

NSAID compounds suitable as one or more constituent moieties in the present invention include: acetaminophen, aspirin, choline magnesium trisalicylate, diclofenac, diflunisal, etodolac, fenoprofen, flurbiprofen, ibuprofen, indomethacin, ketorolac, ketoprofen, meclofenamic acid, mefenamic acid, naproxen, nahumstone, nabumetone, oxaprozin, piroxicam, phenylbutazone, sulindac, and tolmetin, or a pharmaceutically acceptable salt, prodrug, or active metabolite thereof. Each of the foregoing NSAID compounds possesses at least one functional group capable of forming a direct or indirect bond to another moiety having one or more functional groups, and all are thus capable of being linked to one or more of the same NSAID, a different NSAID, or a different pharmaceutically active moiety. Preferred NSAIDs for making codrugs according to the present invention are diclofenac, flurbiprofen, naproxen, and ketoprofen. Preferred salts include sodium and potassium salts.

Suitable analgesic compounds for use as one or more constituent moieties according to the present invention include: benzodiazepam, buprenorphine, butorphanol, codeine, desmorphine, dezocine, dihydromorphine, dimepbeptanol, eptazocine, ethylmorphine, fentanyl, glafenine, hydromorphone, isoladol, ketobenidone, p-lactophetide, levorphanol, lidocaine, moptazinol, metazocin, meperidine, methadone, metopon, morphine, nalbuphine, nalmefene, nalorphine, naloxone, norlevorphanol, normorphine, oxycodone, oxymorphone, pentazocine, phenperidine, phenylramidol, propoxyphene, tramadol, and viminol, or a pharmaceutically acceptable salt, ester, or prodrug thereof. Each of these analgesic compounds above possesses one or more functional groups as defined above, and all are analgesics capable of being linked to one or more of the same analgesic, a different analgesic, or a different pharmaceutically active moiety.

Antiandrogen compounds suitable as one of more constituent moieties in the present invention include luteinizing hormone-releasing hormone (LHRH) agonists or progestational agents, bicalutamide, bifluranol, cyproterone, flutamide, nilutamide, osaterone, oxendolone, etc., or a pharmaceutically salt, ester, or prodrug thereof. Each of these antiandrogen compounds above possesses one or more functional groups as defined above, and all are antiandrogens capable of being linked to one or more of the same antiandrogen, a different antiandrogen, or a different pharmaceutically active moiety.

Alpha-blocker compounds suitable as one of more constituent moieties in the present invention include naftopidol and analogs of phenoxybenzamine and prazosin, or a pharmaceutically acceptable salt or prodrug thereof. Each of these alpha-blocker compounds above possesses one or more functional groups as defined above, and all are alpha-blockers capable of being linked to one or more of the same alpha-blocker, a different alpha-blocker, or a different pharmaceutically active moiety.

Anti-cholinergic compounds suitable as one or more constituent moieties in the present invention include biperiden, procyclidin, trihexylphenidyl hydrochloride, atropine, ipratropium bromide, oxitropium bromide, etc., or a pharmaceutically acceptable salt or prodrug thereof. Each of these anti-cholinergic compounds above possesses one or more functional groups as defined above, and all are anti-cholinergics capable of being linked to one or more of the same anti-cholinergic, a different anti-cholinergic, or a different pharmaceutically active moiety.

Adrenergic compounds suitable as one of more constituent moieties in the present invention include acebutolol, atenolol, betaxolol, timolol, propanolo, etc., or a pharmaceutically acceptable salt or prodrug thereof. Each of these adrenergic compounds above possesses one or more functional groups as defined above, and all are adrenergics capable of being linked to one or more of the same adrenergic, a different adrenergic, or a different pharmaceutically active moiety.

Local anesthetic compounds suitable as one of more constituent moieties in the present invention include ambucaine, benzocaine, butamben, procaine, oxybuprocaine, tetracaine, etc., or a pharmaceutically acceptable salt or prodrug thereof. Each of these local anesthetic compounds above possesses one or more functional groups as defined above, and all are local anesthetics capable of being linked to one or more of the same local anesthetic, a different local anesthetic, or a different pharmaceutically active moiety.

In particular embodiments according to the present invention, a therapeutically effective amount of a biologically active moiety, salt, or composition according to the present invention will deliver a local amount for at least 24 hours, and even more preferably may be for at least 72 hours, 100, 250, 500 or even 750 hours. In some embodiments, a local amount is delivered over at least one week, more preferably two weeks, or even more preferably at least three weeks. In certain embodiments, a local amount is delivered over at least one month, more preferably two months, and even more preferably six months.

In particular embodiments according to the present invention, a therapeutically effective amount of a biologically active moiety, salt, or composition according to the present invention will deliver a locally cytotoxic amount of an antiproliferative agent for at least 24 hours, and even more preferably may be for at least 72 hours, 100, 250, 500 or even 750 hours. In some embodiments, a locally cytotoxic amount is delivered over at least one week, more preferably two weeks, or even more preferably at least three weeks. In certain embodiments, a locally cytotoxic amount is delivered over at least one month, more preferably two months, and even more preferably six months.

In some embodiments according to the present invention, a therapeutically effective amount of a biologically active moiety, salt, or composition according to the present invention will deliver a locally apoptotic amount of an antiproliferative agent for at least 24 hours, and even more preferably may be for at least 72 hours, 100, 250, 500 or even 750 hours. In some embodiments, a locally apoptotic amount is delivered over at least one week, more preferably two weeks, or even more preferably at least three weeks. In certain embodiments, a locally apoptotic amount is delivered over at least one month, more preferably two months, and even more preferably six months.

In some embodiments according to the present invention, a therapeutically effective amount of a biologically active moiety, salt, or composition according to the present invention will deliver a locally anti-inflammatory amount of an antiproliferative agent for at least 24 hours, and even more preferably may be for at least 72 hours, 100, 250, 500 or even 750 hours. In some embodiments, a locally anti-inflammatory amount is delivered over at least one week, more preferably two weeks, or even more preferably at least three weeks. In certain embodiments, a locally anti-inflammatory amount is delivered over at least one month, more preferably two months, and even more preferably six months.

The codrugs may be used for treating tumors in some embodiments. The codrugs may release locally therapeutic levels of antiproliferative moieties while, at the same time, releasing locally effective levels of corticosteroid moieties. The codrugs thus treat tumors while simultaneously reducing the inflammation, and in some cases, the pain associated with tumors. This dual action increases the efficacy of the codrugs by improving patient tolerance of the antiproliferative therapy. The dual action also may, in some cases, reduce diffusive efflux multiple drug resistance by reducing inflammation and the associated elevated fluid pressure in the vicinity of the tumor.

IV. Exemplary Methods

The present invention also provides methods for treating a proliferative disease. A method according to the present invention is useful for treating a cancerous or benign lesion, such as a solid tumor. Cancers treatable with one or more biologically active moieties according to the present invention include cervical cancer, uterine cancer, ovarian cancer, prostate cancer, pancreatic cancer, and lymphomas, including Hodgkins and non-Hodgkins lymphomas. Other proliferative diseases treatable with devices according to the present invention include benign prostatic hypertrophy (BPH). A preferred method of treatment according to the present invention is treatment of BPH or prostate cancer, optionally in combination therapy with radiotherapy.

In certain embodiments, the method comprises administering to an individual, such as a human or non-human mammal, at least one therapeutically effective dose of a codrug, a salt thereof, or a composition comprising a codrug. A therapeutically effective amount of a codrug, salt, or composition according to the present invention is an amount that, when administered in a course of treatment, is sufficient to bring about one or more of the following effects: halt the growth or spread of a neoplastic disease, prevent metastasis of a neoplastic lesion, produce a cytotoxic effect in a neoplastic lesion, induce apoptosis in cancerous or pre-cancerous neoplastic cells, reduce or prevent local or systemic inflammation, or reduce pain associated with a neoplastic lesion. In certain embodiments according to the present invention, a therapeutically effective dose is an amount of a codrug, salt, or composition according to the present invention that releases sufficient antiproliferative agent in sufficient concentration over a period of time sufficient to produce a cytotoxic effect in the target neoplastic lesion.

The present invention includes methods for treatment of a patient in need of such treatment. The patient may be of any mammalian species, especially human. Veterinary patients include species of dogs, cats, horses, cattle, and swine. The need for treatment is determined by a skilled physician or veterinarian based upon the symptoms presented by the patient.

Actual dosage levels of the active ingredients in the pharmaceutical compositions of this invention may be varied so as to obtain an amount of the active ingredient which is effective to achieve the desired therapeutic response for a particular patient and composition, without being toxic to the patient.

The selected dosage level will depend upon a variety of factors including the activity of the constituent drugs of the particular codrug employed in a drug delivery device of the present invention, or the ester, salt, or amide thereof, the time of administration, the rate of excretion of the particular codrug (and/or its constituent drugs) being employed, the duration of the treatment, other biologically active moieties, materials used in combination with the particular codrug employed, the age, species, sex, weight, condition, general health and prior medical history of the patient being treated, and like factors well known in the medical arts.

A physician or veterinarian of ordinary skill in the art can readily determine and prescribe the effective amount of the codrug required. For example, the physician or veterinarian could start doses of the codrugs of the invention employed in the drug delivery device at levels lower than that required in order to achieve the desired therapeutic effect and gradually increase the dosage until the desired effect is achieved.

A method of treatment according to the present invention can be used to treat a number of diverse physical ailments. In this context, the terms “treat”, “treating”, and “treatment” include alleviation of one or more symptoms, reduction in the rate of progress of a progressive disease state, induction of remission of a disease state, and cure. In some embodiments according to the present invention, the symptoms alleviated include pain, inflammation, itching, numbness, nausea, voiding, incontinence, and vomiting, or a combination of two or more of these symptoms. In some embodiments according to the present invention, the disease state to be treated is a proliferative disease, such as a neoplastic disease, such as melanoma, Hodgkins disease, non-Hodgkins lymphoma, or cancer. In some embodiments, the method according to the present invention causes a reduction in symptoms, such as pain, and/or slows or ceases progress of the disease by slowing or halting cell division of the disease cells, and/or induces remission of the disease by selectively killing disease cells or by slowing disease cell proliferation sufficiently to allow the patient's immune system to combat the disease.

A method of treatment according to the present invention may be used to treat various symptoms and disease states, such as pain, inflammation, and itching, either by themselves or concomitant with an underlying disease condition. Other disease states that may be treated by a method according to the present invention include proliferative diseases, such as melanoma, lymphomas, sarcomas, and carcinomas, etc.

A device or a method of treatment according to the present invention may be used in conjunction with other treatments, such as radiation therapy, chemotherapy, transurethral resection of the prostrate, transurethral microwave therapy, transurethral thermal therapy, laser ablation, etc. These treatments may have synergistic or complementary effects.

V. Exemplary Compositions

Drug delivery devices according to the present invention are suitable for implantation, for example, surgical implantation, implanted using needles, cannulas, catheters, etc. It may be advantageous to formulate the subject compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are spacers, pellets, and segregated multiples thereof.

Some embodiments of a drug delivery device according to the present invention may conveniently be presented in unit dosage forms and may be prepared by any methods well known in the art. The amount of active ingredient which can be combined with a material to produce a single dosage form will generally be that amount of the codrug which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these devices include bringing into association a codrug of the present invention with a vehicle material and, optionally, one or more accessory ingredients. In some embodiments, the formulations are prepared by uniformly and intimately bringing into association a codrug of the present invention with liquid vehicles, or finely divided solid vehicles, or both, and then, if necessary, shaping the product.

In certain embodiments according to the present invention, the drug delivery devices comprise codrugs, wherein said codrugs comprise a residue of an antiproliferative compound or salt thereof linked directly or indirectly to a residue of a corticosteroid antiinflammatory agent or salt thereof, in an amount convenient for therapeutic administration, optionally in admixture with one or more pharmaceutically acceptable adjuvants, excipients, diluents, carriers, or dispersants. The adjuvant, excipient, diluent, carrier, or dispersant will vary depending upon the condition to be treated, the structure of the codrug, etc.

The codrugs of the present invention may also be provided in the form of prodrugs, e.g., to protect a biologically active moiety from being altered while passing through a hostile environment. Prodrugs can be prepared by forming covalent linkages between the biologically active moiety and a modifier. See, for example, Balant at al., Eur. J. Drug Metab. Pharmacokinetics, 1990, 15 (2), 143-153. The linkage is usually designed to be cleaved under defined circumstances, e.g., pH changes or exposure to specific enzymes. The covalent linkage of the biologically active moiety to a modifier essentially creates a new molecule with new properties such as an altered log P value and/or as well as a new spatial configuration. The new molecule can have different solubility properties and be less susceptible to enzymatic digestion. For general references on prodrug design and preparation, see: Bundraard, Design of Prodrugs, Elsevier Science Pub. Co., N.Y. (1985), and Prodrugs as Novel Drug Delivery Systems Symposium, 168^{th} Annual Meeting, American Chemical Society, Atlantic City, N.J., Eds. T. Higuchi and V. Stella, ACS Symposium Series 14, 1975, which are herein incorporated by reference.

Prodrugs of amine-containing moieties are well known in the art and have been prepared, e.g., by reacting the amine moiety of a moiety with a carboxylic acid, acid chloride, chloroformate, or sulfonyl chloride modifiers, and the like, resulting in the formation of amides, sulfonamides, carboxyamides, carbamates, and similar compounds. See, for example, Abuchowski et al., J. Biol. Chem. 1977, 252, 3578-358; Senter et al., J. Org. Chem., 1990, 55, 2975-2978; Amsberry et al., J. Org. Chem., 1990, 55, 5867-5877; Klotz, Clin. Pharmacokinetics, 1985, 10, 285-302, which are herein incorporated by reference. Similar and other protocols may be followed for the formation of prodrugs of the codrugs of the present invention.

The proportion of codrug in the drug delivery devices can vary from between about 0.01 wt. % to about 100 wt. %, more preferably from about 0.1 wt. % to about 99.9 wt. %, and especially from about 1.0 wt. % to about 99.0 wt. %.

Codrugs according to the present invention may be prepared in free form, or may be prepared as salts, such as mineral acid, carboxylic acid, ammonium hydroxide or amine salts thereof. Codrugs according to the present invention may be prepared as amorphous or crystalline forms, and may be in the form of anhydrates or hydrates. Codrugs according to the present invention may be present as prodrugs, such as esters. In each of these cases, the critical feature is that a codrug according to the present invention be stable under some conditions other than physiologic conditions, and be capable of decomposing under physiologic conditions to form first and second constituent moieties, which moieties may be the same or different, as discussed above.

As set out above, certain embodiments of the present codrugs may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of codrugs of the present invention. These salts can be prepared in situ during the final isolation and purification of the codrugs of the invention, or by separately reacting a purified codrug of the invention in its free base form with a suitable organic or inorganic acid, and isolating the salt thus formed. Representative salts include the hydrobromide, hydrochloride, sulfate, bisulfate, formate, borate, phosphate, nitrate, acetate, valerate, oleate, palmitate, stearate, laurate, benzoate, lactate, phosphonate, tosylate, citrate, maleate, fumarate, succinate, tartrate, naphthylate, mesylate, glucoheptonate, lactobionate, and laurylsulphonate salts and the like. (See, for example, Berge et al. (1977) “Pharmaceutical Salts”, J. Pharm. Sci. 66: 1-19).

The pharmaceutically acceptable salts of the subject codrugs include the conventional nontoxic salts or quaternary ammonium salts of the codrugs, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the codrugs of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of codrugs of the present invention. These salts can likewise be prepared in situ during the final isolation and purification of the codrugs, or by separately reacting the purified codrug in its free acid form with a suitable base, such as the hydroxide, carbonate or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers, surfactants, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The present invention provides a drug delivery system that can provide various release profiles, e.g., varying doses and/or varying lengths of time. The present invention thereby addresses the need for an insertable or implantable drug delivery system that provides controlled time-release kinetics of drug, particularly in the vicinity of a desired locus of drug activity, while avoiding complications associated with prior art devices.

A device of the present invention may include a polymer and a codrug having a low solubility dispersed in the polymer. The polymer may be permeable to the codrug or may gradually decompose or degrade in vivo, and is preferably essentially non-release rate limiting with respect to the rate of release of the codrug from the polymer, and provides sustained release of the drug.

Once administered, in some embodiments, the device gives a continuous supply of the codrug to the desired locus of activity without necessarily requiring additional invasive penetrations into these regions. Instead, the device may remain in the body and serve as a continuous source of the codrug to the affected area. In some embodiments, the device according to the present invention permits prolonged release of drugs over a specific period of days, weeks, months (e.g., about 3 months to about 6 months) or years (e.g., about 1 year to about 20 years, such as from about 5 years to about 10 years) until the codrug is used up.

In some embodiments, the codrugs are slowly dissolved in physiologic fluids, but upon dissolution, are relatively quickly dissociated into at least one pharmaceutically active compound. In some embodiments, the dissolution rate of the codrug is in the range of about 0.001 μg/day to about 10 μg/day. In certain embodiments, the codrugs have dissolution rates in the range of about 0.01 to about 1 μg/day. In particular embodiments, the codrugs have dissolution rates of about 0.1 μg/day.

The low-solubility pharmaceutical codrug may be incorporated into a biocompatible (i.e., biologically tolerated) polymer vehicle. In some embodiments according to the present invention, the low-solubility pharmaceutical codrug is present as a plurality of granules dispersed within the polymer vehicle. In such cases, it is preferred that the low-solubility pharmaceutical codrug be relatively insoluble in the polymer vehicle, however the low-solubility pharmaceutical codrug may possess a finite solubility coefficient with respect to the polymer vehicle and still be within the scope of the present invention. In either case, the polymer vehicle solubility of the low-solubility pharmaceutical codrug should be such that the codrug will disperse throughout the polymer vehicle.

In some embodiments according to the present invention, the low-solubility pharmaceutical codrug is dissolved within the polymer vehicle. In such cases, it is preferred that the polymer vehicle be a relatively non-polar or hydrophobic polymer which acts as a good solvent for the relatively hydrophobic low-solubility pharmaceutical codrug. In such cases, the solubility of the low-solubility pharmaceutical codrug in the polymer vehicle should be such that the codrug will dissolve thoroughly in the polymer vehicle, being distributed homogeneously throughout the polymer vehicle.

In certain embodiments, a polymer useful according to the present invention comprises any biologically tolerated polymer that is permeable to the codrug and, yet has a permeability such that it is not the principal rate-determining factor in the rate of release of the codrug from the polymer.

In some embodiments according to the present invention, the polymer is non-bioerodible. Examples of non-bioerodible polymers useful in the present invention include poly(ethylene-co-vinyl acetate) (EVA), polyvinylalcohol and polyurethanes, such as polycarbonate-based polyurethanes. In other embodiments of the present invention, the polymer is bioerodible. Examples of bioerodible polymers useful in the present invention include polyanhydride, polylactic acid, polyglycolic acid, polyorthoester, polyalkylcyanoacrylate or derivatives and copolymers thereof. The skilled artisan will recognize that the choice of bioerodibility or non-bioerodibility of the polymer depends upon the final physical form of the system, as described in greater detail below. Other exemplary polymers include polysilicone and polymers derived from hyaluronic acid. The skilled artisan will understand that the polymer according to the present invention is prepared under conditions suitable to impart permeability such that it is not the principal rate-determining factor in the release of the low solubility codrug from the polymer.

Moreover, suitable polymers include naturally occurring (collagen, hyaluronic acid, etc.) or synthetic materials that are biologically compatible with bodily fluids and mammalian tissues, and essentially insoluble in bodily fluids with which the polymer will come in contact. In addition, the suitable polymers essentially prevent interaction between the low solubility codrug dispersed/suspended in the polymer and proteinaceous components in the bodily fluid. The use of rapidly dissolving polymers or polymers highly soluble in bodily fluid or which permit interaction between the low solubility codrug and proteinaceous components are to be avoided in certain instances since dissolution of the polymer or interaction with proteinaceous components would affect the constancy of drug release.

Other suitable polymers include polypropylene, polyester, polyvinyl alcohol (PVA), polyethylene vinyl acetate (EVA), polyethylene oxide (PEO), polypropylene oxide, polycarboxylic acids, polyalkylacrylates, cellulose ethers, silicone, poly(dl-lactide-co glycolide), various Eudragits (for example, NE30D, RS PO and RL PO), polyalkyl-alkyacrylate copolymers, polyester-polyurethane block copolymers, polyether-polyurethane block copolymers, polydioxanone, poly-(β-hydroxybutyrate), polylactic acid (PLA), polycaprolactone, polyglycolic acid, and PEO-PLA copolymers.

A coating of the present invention may be formed by mixing one or more suitable monomers and a suitable low-solubility pharmaceutical codrug, then polymerizing the monomer to form the polymer system. In this way, the codrug is dissolved or dispersed in the polymer. In other embodiments, the codrug is mixed into a liquid polymer or polymer dispersion and then the polymer is further processed to form the inventive coating. Suitable further processing may include crosslinking with suitable crosslinking codrugs, further polymerization of the liquid polymer or polymer dispersion, copolymerization with a suitable monomer, block copolymerization with suitable polymer blocks, etc. The further processing traps the drug in the polymer so that the drug is suspended or dispersed in the polymer vehicle.

Any number of non-erodible polymers may be utilized in conjunction with the drug combination. Film-forming polymers that can be used for coatings in this application can be absorbable or non-absorbable and must be biocompatible to minimize irritation to the surrounding tissue. The polymer may be either biostable or bioabsorbable depending on the desired rate of release or the desired degree of polymer stability, but a bioabsorbable polymer may be preferred since, unlike biostable polymer, it will not be present long after implantation to cause any adverse, chronic local response. Further, the bioabsorbable polymer tends not to migrate.

Suitable film-forming bioabsorbable polymers that could be used include polymers selected from aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-, 1-, and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include polymers as described by Kemnitzer and Kohn, in the Handbook of Biodegradable Polymers, edited by Domb, Kost, and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30 (1), page 498, 1989 (e.g., PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C₆H₄—O—(CH₂)ₘ—O—C₆H₄—COOH where m is an integer in the range of from 2 to 8 and copolymers thereof with aliphatic alpha-omega diacids of up to 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference). Film-forming polymeric biomolecules for the purpose of this invention include naturally occurring materials that may be enzymatically degraded in the human body or are hydrolytically unstable in the human body such as fibrin, fibrinogen, collagen, elastin, and absorbable biocompatible polysaccharides such as chitosan, starch, fatty acids (and esters thereof), glucoso-glycans, and hyaluronic acid.

Suitable film-forming biostable polymers with relatively low chronic tissue response, such as polyurethanes, silicones, poly(meth)acrylates, polyesters, polyalkyl oxides (polyethylene oxide), polyvinyl alcohols, polyethylene glycols, and polyvinyl pyrrolidone, as well as, hydrogels such as those formed from crosslinked polyvinyl pyrrolidinone and polyesters could also be used.

In certain embodiments, multiple coatings can be used. For instance, the various coatings can differ in the concentration of codrug, the identity of the codrugs (active ingredients, linkers, etc), the characteristics of the polymer matrix (composition, porosity, etc) and/or the presence of other drugs or release modifiers.

U.S. Pat. No. 5,773,019, U.S. Pat. No. 6,001,386, and U.S. Pat. No. 6,051,576 disclose implantable controlled-release devices and drugs and are incorporated in their entireties herein by reference.

In some embodiments according to the invention, the device comprises a polymer that is relatively rigid. In other embodiments, the device comprises a polymer that is soft and malleable. In still other embodiments, the device includes a polymer that has an adhesive character. Hardness, elasticity, adhesive, and other characteristics of the polymer are widely variable, depending upon the particular final physical form of the device, as discussed in more detail below.

Embodiments of the device according to the present invention take different forms. In some embodiments, the device comprises the low solubility codrug, i.e., the codrug suspended or dispersed in the polymer. In certain other embodiments, the device comprises a codrug and a solid polymer, which is adapted to be injected via a syringe into a body. In other embodiments according to the present invention, the device comprises a codrug and a soft-flexible polymer, which is adapted to be inserted or implanted into a body by a suitable surgical method. In still further embodiments according to the present invention, the device comprises a hard, solid polymer, which is adapted to be inserted or implanted into a body by a suitable surgical method.

In some embodiments according to the present invention wherein the polymer is poorly permeable and bioerodible, the rate of bioerosion of the polymer is advantageously sufficiently slower than the rate of drug release so that the polymer remains in place for a substantial period of time after the drug has been released, but is eventually bioeroded and reabsorbed into the surrounding tissue.

In other embodiments according to the present invention, the rate of bioerosion of the polymer is advantageously on the same order as the rate of drug release. For instance, the polymer advantageously may bioerode at such a rate that the surface area of the codrug that is directly exposed to the surrounding body tissue remains substantially constant over time.

In other embodiments according to the present invention, the polymer vehicle is permeable to water in the surrounding tissue, e.g., in blood plasma. In such cases, water solution may permeate the polymer, thereby contacting the low-solubility pharmaceutical codrug. The rate of dissolution may be governed by a complex set of variables, such as the polymer's permeability, the solubility of the low-solubility pharmaceutical codrug, the pH, ionic strength, and protein composition, etc., of the physiologic fluid. In certain embodiments, however the permeability may be adjusted so that the rate of dissolution is governed primarily, or in some cases practically entirely, by the solubility of the low-solubility pharmaceutical codrug in the ambient liquid phase.

In some embodiments according to the present invention, the device according to the present invention is advantageously a solid device of a shape and form suitable for implantation.

As used in regard to the low-solubility pharmaceutical codrug, the term “low-solubility” relates to the solubility of a pharmaceutical codrug in biological fluids, such as blood plasma, lymphatic fluid, peritoneal fluid, etc. In general, “low-solubility” means that the pharmaceutical codrug is only very slightly soluble in aqueous solutions having pH in the range of about 5 to about 8, and in particular to physiologic solutions, such as blood, blood plasma, etc. Some low-solubility codrugs according to the present invention will have solubilities of less than about 1 mg/ml, less than about 100 μg/ml, preferably less than about 20 μg/ml, more preferably less than about 15 μg/ml, and more preferably less than about 10 μg/ml. Solubility is measured in water at a temperature of 25° C. according to the procedures set forth in the 1995 USP, unless otherwise stated. This includes compounds which are slightly soluble (about 10 mg/ml to about 1 mg/ml), very slightly soluble (about 1 mg/ml to about 0.1 mg/ml) and practically insoluble or insoluble compounds (less than about 0.01 mg/ml).

Equivalents

Those skilled in the art will recognize, or be able to ascertain, using no more than routine experimentation, numerous equivalents to the specific biologically active moieties, methods, diluents, polymers, and salts described herein. Such equivalents are considered to be within the scope of this invention.

Exemplification

The present invention may be further appreciated upon consideration of the following illustrative and non-limiting examples.

The foregoing written description is intended to illustrate the principles of the invention, and is not intended to be limiting. One skilled in the art will readily appreciate that other embodiments are possible within the scope of the present invention, as described above and in the following claims.

All references cited herein, including patents, patent applications and non-patent literature, are explicitly incorporated herein by reference.

The following examples of codrugs usable in the present invention are read in the context of U.S. Pat. No. 6,051,576, which details how the data of the examples and the associated schemes are interpreted.

### EXAMPLE 1

### Codrug (2) of Flurbiprofen with 5-Fu (Scheme 1)

Bis(hydroxymethyl) 5-fluorouracil (1), (0.17 g) was dissolved in 3 mL of anhydrous acetonitrile under argon. To this stirred solution at room temperature was added triethylamine (0.195 mL) followed by acid chloride of flurbiprofen (0.282 g). The cloudy mixture was stirred at room temperature overnight, diluted with 10 mL of dichloromethane, washed with 1 M HCl, aq. sodium bicarbonate, water, brine and dried over sodium sulfate. The oily residue after solvent evaporation was purified by column chromatography on silica gel using chloroform-methanol 100:1 to afford 0.19 g of the codrug (2) as a colorless crystalline solid. ¹H NMR (CDCl₃), 1.50 (d, 3H), 3.94 (q, 1H), 5.72 (s, 2H), 7.22 (dd, 2H), 7.37-7.57 (m, 6H), 7.92 (d, 1H).

### EXAMPLE 2

### Codrug (3) of Indomethacin with 5-FU (Scheme 1)

Bis(hydroxymethyl) 5-fluorouracil (0.39 g) was dissolved in 15 mL of anhydrous acetonitrile under argon. To this stirred solution was added indomethacin (0.81 g) followed by DCC (0.46 g) and catalytic amount of DMAP. The resulting yellow suspension was stirred at room temperature overnight and evaporated to dryness under vacuum. The solid residue was then purified by column chromatography on silica gel in chloroform-methanol 100:2 to afford 0.63 g of the codrug (3). ¹H NMR (DMSO d₆), 2.20 (s, 3H), 3.72 (s, 3H), 5.60 (s, 2H), 6.68 (m, 1H), 6.91 (d, 1H), 7.00 (d, 1H), 7.63 (s, 5H), 8.12 (d, 1H).

### EXAMPLE 3

### Codrug (4) of Sulindac with 5-FU (Scheme 1)

Bis(hydroxymethyl) 5-fluorouracil (0.40 g) was dissolved in 5 mL of anhydrous acetonitrile under argon. To this stirred solution was added sulindac (0.75 g) followed by EDCI (0.40 g) and catalytic amount of DMAP. The orange mixture soon turned homogenous and it was kept overnight at room temperature in darkness. Evaporation of the solvent left the crude residue which was dissolved in dichloromethane (20 mL) and washed twice with water, once with saturated sodium bicarbonate, water and brine. The extract was dried over sodium sulfate, evaporated and purified by column chromatography on silica gel using chloroform-methanol 30:1 as solvent system to yield 0.69 g of codrug (4). ¹H NMR (CDCl₃), 2.20(s, 3H), 2.82 (s, 3H), 3.64 (s, 2H), 5.66 (s, 2H), 6.57 (m, 1H), 6.81 (dd, 1H), 7.15 (m, 2H), 7.51 (d, 1H), 7.70 (dd, 4H).

### EXAMPLE 4

### Codrug (5) of Diclofenac with 5-FU (Scheme 1)

To a stirred solution of bis(hydroxymethyl) 5-fluorouracil (0.147 g) in anhydrous acetonitrile (2 mL) at 0-5° C. under argon was added diclofenac (0.148 g) followed by EDCI (0.115 g) and DMAP (4 mg). The mixture soon turned homogenous and it was left in a refrigerator overnight. The solvent was evaporated under vacuum, the residue dissolved in ethyl acetate, washed three times with water, once with brine and dried over anhydrous sodium sulfate. The residue after solvent evaporation was purified by flash chromatography in chloroform-methanol 40:1 to afford 0.16 g of the codrug (5) as colorless foam. ¹H NMR (CDCl₃), 3.87 (s, 2H), 5.67 (s, 2H), 6.45 (s, 1H), 6.55 (d, 1H), 6.92-7.28 (m, 5H), 7.33 (d, 2H), 7.57 (d, 1H), 9.50 (br. s, 1H).

### EXAMPLE 5

### Codrug (6) of 5-FU with Naproxen (Scheme 1)

Bis(hydroxymethyl) 5-fluorouracil (1) prepared from 6.21 g of 5-FU and 8.51 g of 37% formalin was dissolved in a mixture of dichloromethane (45 mL) and acetonitrile (15 mL). Anhydrous triethylamine (6.66 mL) was added and the resulting solution was cooled in an ice bath under argon. Naproxen acid chloride (5.94 g) was dissolved in 30 mL of anhydrous dichloromethane and this solution was added dropwise to the reaction mixture at 0-5° C. The resulting pale yellow homogenous solution was kept at room temperature overnight. The mixture was diluted with 100 mL of dichloromethane, washed with 1M HCl, twice with saturated sodium bicarbonate, water, brine and dried over anhydrous sodium sulfate. The crude product after solvent evaporation was recrystalized from absolute ethanol to yield 7.7 g of codrug (2) as colorless powder. ¹H NMR (CDCl₃), 1.60 (d, 3H), 3.84 (q, 1H), 3.86 (s, 3H), 5.60 (s, 2H), 7.06 7.70 (m, 7H), 9.05 (s, 1H).

### EXAMPLE 6

### Codrug (7) of Aspirin with 5-FU (Scheme 1)

Bis(hydroxymethyl) 5-fluorouracil (0.38 g) was dissolved in 8 mL of anhydrous acetonitrile under argon. The stirred solution was cooled in an ice bath and pyridine (0.2 mL) was added followed by solid acetylosalicyloyl chloride (0.437 g). The homogenous solution was left protected from moisture at room temperature overnight. The solvent was evaporated under vacuum; the residue was dissolved in 40 mL of dichloromethane, washed with saturated sodium bicarbonate solution, water, brine and dried over sodium sulfate. Solvent evaporation gave yellow oily residue which was purified by column chromatography on silica gel in chloroform-methanol 40:1 to provide 0.32 g of the codrug (7) as pale yellow oil. ¹H NMR (CDCl₃), 2.37 (s, 3H), 5.83 (s, 2H), 7.12 (m, 1H), 7.34 (m, 1H), 7.62 (m, 1H), 7.72 (d, 2H), 8.05 (dd, 1H).

### EXAMPLE 7

### Codrug (8) of Naproxen with 5-FU (Scheme 2)

To a stirred suspension of 5-FU (0.13 g) in anhydrous acetonitrile (4 mL) at 0-5° C. under argon was added triethylamine (0.070 mL) followed by naproxen acid chloride (0.124 g). The cloudy mixture was stirred at room temperature overnight, filtered and evaporated to dryness to yield 0.28 g of crude product. It was dissolved in ethyl acetate, filtered and evaporated to afford 0.21 g of the codrug (8). ¹H NMR (CDCl₃), 1.63 (d, 3H), 3.91 (s, 3H), 3.92 (q, 1H), 7.09 7.36 (m, 6H), 8.23 (d, 1H).

### EXAMPLE 8

### Canine Study Using 5-FU/FA Codrug

A canine study was performed using a codrug of 5-FU and fluocinolone acetonide (Fa) to evaluate the release rate of the codrug in prostate tissue. The results of that study are detailed in FIGS. 1-3. Two 5 mg pellets of the codrug were implanted in the prostate, and the animals were humanely sacrificed on postoperative days, as indicated. The prostates were removed and levels of the codrug in the pellets determined. This study demonstrates that efficacious levels of 5-FU and FA can be delivered locally to the prostrate using a codrug thereof.

TABLE 1
Dog Prostate Result (5 mg implants)

| (Dog) | Date | Codrug | | 5-FU + FA |
|---|---|---|---|---|
| Animal | Animal | Remaining | | Mean Remaining |
| No. | Sacrificed | (mg) | | (mg) |
| 10226 | (3 days) | 1.76 | (pellet 1) | 3 days: 4.26 |
| | | 5.05 | (pellet 2) | |
| 10227 | (3 days) | 5.17 | (pellet 1) | |
| | | 5.04 | (pellet 2) | |
| 10224 | (7 days) | 3.52 | (pellet 1) | |
| | | 5.10 | (pellet 2) | |
| 10228 | (7 days) | 2.08 | (pellet 1) | 7 days: 3.64 |
| | | 3.85 | (pellet 2) | |
| 10190 | (28 days) | 4.27 | (pellet 1) | |
| | | 4.24 | (pellet 2) | |
| 10220 | (28 days) | 4.04 | (pellet 1) | 28 days: 4.09 |
| | | 3.50 | (pellet 2) | |
| 10218 | (28 days) | 4.14 | (pellet 1) | |
| | | 4.18 | (pellet 2) | |
| 10219 | (28 days) | 4.12 | (pellet 1) | |
| | | 4.23 | (pellet 2) | |
| 10214 | (42 days) | 3.50 | (pellet 1) | |
| | | 3.33 | (pellet 2) | |
| 10216 | (42 days) | 3.89 | (pellet 1) | 42 days: 3.68 |
| | | 4.12 | (pellet 2) | |
| 10263 | (60 days) | 0.243 | (pellet 1) | |
| | | 0.534 | (pellet 2) | |
| 10273 | (60 days) | 0.161 | (pellet 1) | |
| | | 0.047 | (pellet 2) | |
| 10274 | (60 days) | 1.270 | (pellet 1) | |
| | | 0.763 | (pellet 2) | |
| 10272 | (90 days) | 0.243 | (pellet 1) | |
| 10286 | (60 days) | 0.070 | (pellet 1) | 90 days: 0.071 |
| | | 0.064 | (pellet 2) | |
| 10264 | (90 days) | 0.092 | (pellet 1) | |

TABLE 2
Mean Remaining of the Codrug:

| | |
|---|---|
| 3 days | 5.09 +/− 0.07 mg |
| 7 days | 3.64 +/− 1.24 mg |
| 28 days | 4.09 +/− 0.25 mg |
| 42 days | 3.68 +/− 0.34 mg |

### EXAMPLE 9

### Rabbit Study Using 5-FU/FA Codrug

A rabbit study was performed using a codrug of 5-FU and FA to evaluate the release rate of the codrug in liver tissue. The results of that study are detailed in FIGS. 4 and 5. Two 5 mg pellets of the codrug were implanted in the liver, and the animals were humanely sacrificed on postoperative days, as indicated. The livers were removed and levels of the codrug in the pellets determined. This study demonstrates that efficacious levels of 5-FU and FA can be delivered locally to other tissues using a codrug thereof, and can achieve zero-order release rates over extremely long time periods.

TABLE 3
Rabbit Study - Liver Result (5 mg pellet)

| Time from | | |
|---|---|---|
| Sacrifice (days) | FA-5-FU Codrug Remaining (mg) | |
| 21 day | 3.12 (pellet 1) | Mean = 2.81 +/− 0.30 mg |
| 21 day | 2.86 (pellet 2) | (3 from 4) |
| 21 day | 2.46 (pellet 3) | |
| 31 day | 2.52 (pellet 1) | Mean = 2.63 +/− 0.13 mg |
| 31 day | 2.58 (pellet 2) | (3 from 6) |
| 31 day | 2.79 (pellet 3) | |
| 53 day | 1.15 (pellet 1) | Mean = 1.29 +/− 0.19 mg |
| 53 day | 1.42 (pellet 2) | (2 from 6) |
| 132 day | 0.44 (pellet 1) | |

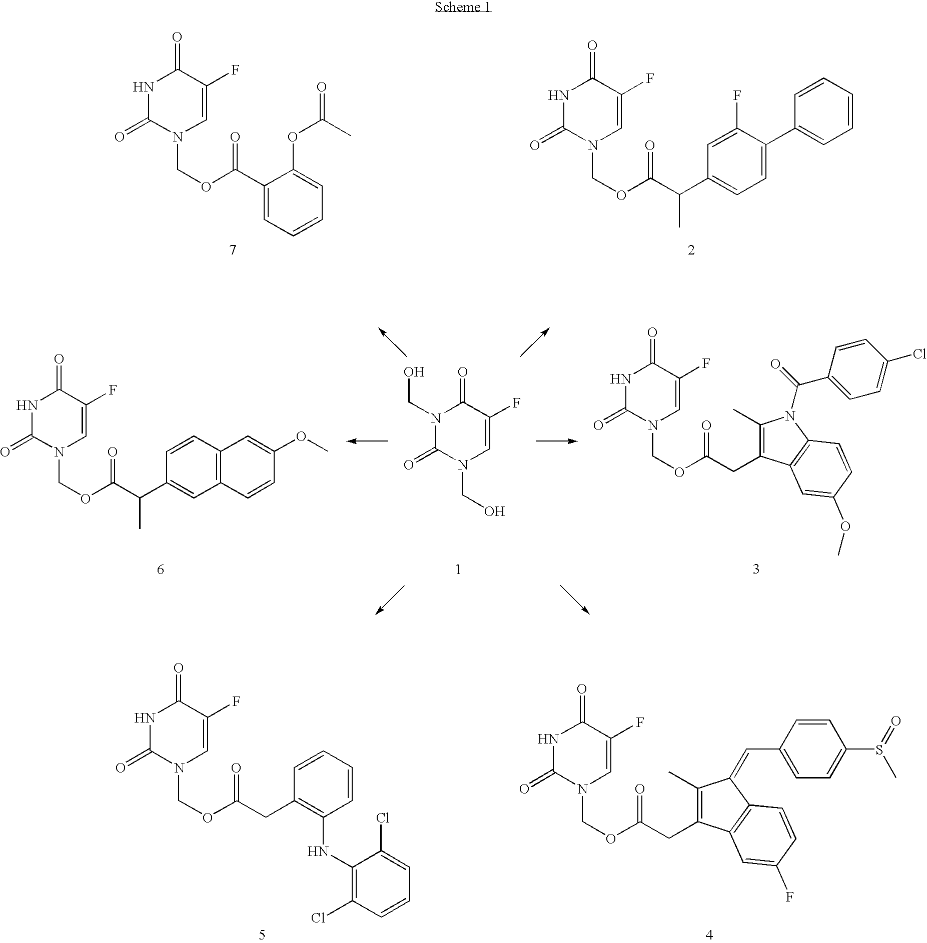

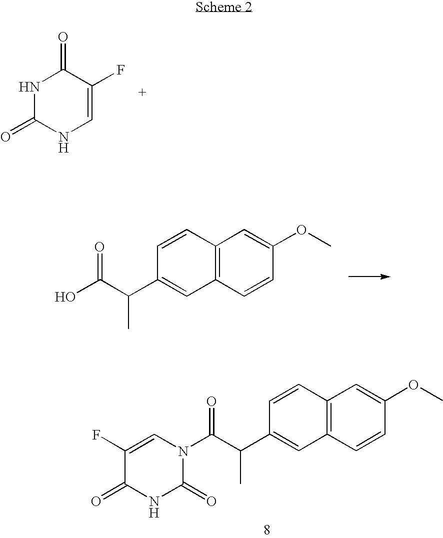

1. A drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device is dimensioned to position two radiation seeds a predetermined distance apart.

2. The drug delivery device according to claim 1, wherein the first constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, adrenergics, purinergics, suppressors of bladder smooth muscle, dopaminergics, local anesthetics, vanilloids, steroids, and anti-cancer agents.

3. The drug delivery device according to claim 2, wherein the second constituent moiety is selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents.

4. The drug delivery device according to claim 1, wherein the first constituent moiety is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or a pharmaceutically acceptable salt thereof.

5. The drug delivery device according to claim 1, wherein the codrug has the structural formula:
R₁-L-(R₂)ₙ
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
n is an integer of from 1 to 4; and
L is selected from a direct bond and a linking group.

6. The drug delivery device according to claim 1, wherein the codrug has the structural formula:
R₁-(L-R₂)ₙ
wherein the first constituent moiety is R₁;
the second constituent moiety is R₂;
R₁ and R₂ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
n is an integer of from 1 to 4; and
L is selected from a direct bond and a linking group.

7. The drug delivery device according to claim 1, wherein the codrug has the structural formula:
(R₁-L)ₘR₂(L₂-R₃)ₙ
wherein the first constituent moiety is R₁;
the second constituent moiety is R₂;
R₁, R₂, and R₃ each represent, independently, a residue of a compound selected from analgesic compounds, anti-inflammatory steroidal compounds (corticosteroids), non-steroidal anti-inflammatory compounds (NSAIDs), antibiotic compounds, anti-fungal compounds, antiviral compounds, antiproliferative compounds, immunomodulatory compounds, cell transport/mobility impeding agents, cytokines, peptides, proteins, alpha-blockers, anti-androgens, anti-cholinergics, antihistaminics, adrenergics, purinergics, suppressors of bladder smooth muscle, dopaminergics, local anesthetics, vanilloids, steroids, and anti-cancer agents;
m is an integer of from 1 to 4;
n is an integer of from 1 to 4; and
L and L₂ are each independently selected a direct bond and a linking group.

8. The drug delivery device according to claim 5, wherein R₂ is a residue of diclofenac, etodolac, ketorolac, indomethacin, sulindac, tolmetin, nabumetone, piroxicam, acetaminophen, fenoprofen, flurbiprofen, ibuprofen, ketoprofen, naproxen, oxaprozin, aspirin, choline magnesium trisalicylate, diflunisal, meclofenamic acid, mefenamic acid, phenylbutazone, or a pharmaceutically acceptable salt thereof.

9. The drug delivery device according to claim 1, wherein the first constituent moiety is a residue of alitretinoin (9-cis-retinoic acid); amifostine; bexarotene (4-[1-(5,6,7,8-tetrahydro-3,5,5,8,8-pentamethyl-2-naphthalenyl) ethenyl]benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-FU; gemcitabine; hydroxyurea; idarubicin; irinotecan; melphalan; methotrexate; mitoxantrone; paclitaxel; pentostatin; streptozocin; temozolomide; teniposide; tomudex; topotecan; valrubicin (N-trifluoroacetyladriamycin-14-valerate); or vinorelbine.

10. The drug delivery device according to claim 1, wherein the first constituent moiety is a residue of: 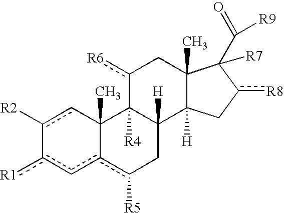
wherein R1 is ═O, —OH, or —(CH₂)₁₋₄Cl;
R2 is H, C₁₋₄alkyl, Cl, or Br;
R4 is H, F, or Cl;
R5 is H, F, Cl, CH₃, or —CHO;
R6 is H, OH, or Cl;
R7 is H, OH, CH₃, O—COCH₃, O(CO)OCH₂CH₃, O—(CO)-2-furanyl, or O—C(O)—(CH₂)₂CH₃;
R8 is H, CH₃, OH, ═CH₂, or together R7 and R8 form, together with the adjacent carbon atoms to which they are attached: 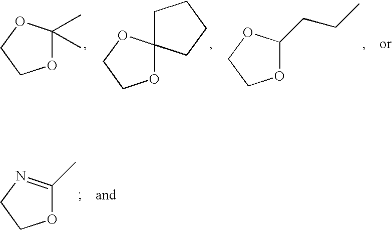
R9 is CH₃, CH₂OH, CH₂O(CO)CH₃, CH₂—O—C₁₋₄alkyl, CH₂Cl, —OCH₂Cl, —CH₂—N—(N′-methyl)piperazinyl, —CH₂—O—(CO)—CH₂—N(Et)₂, ethyl, CH₂SH, CH₂O(CO)C₁₋₄alkyl, CH₂(CO)C(2-propyl)-NH(CO)C₆H₅, or —S—CH₂—F; and
wherein the bonds indicated by 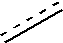
are either double or single bonds.

11. The drug delivery device according to claim 1, wherein the first constituent moiety is residue of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide, or a pharmaceutically acceptable salt thereof.

12. The drug delivery device according to claim 1, further comprising a carrier, an excipient, a solvent, an adjuvant, a diluent, a dispersant, or a surfactant.

13. The drug delivery device according to claim 1, further comprising a biocompatible polymer.

14. The drug delivery device according to claim 13, wherein the polymer comprises polyvinyl alcohol (PVA).

15. The drug delivery device according to claim 13, wherein the codrug, a pharmaceutically acceptable salt, or prodrug thereof, is coated by the biocompatible polymer.

16. The drug delivery device according to claim 13, wherein the codrug, a pharmaceutically acceptable salt, or prodrug thereof, is distributed as particles within the biocompatible polymer.

17. The drug delivery device according to claim 13, wherein the codrug or pharmaceutically acceptable salt or prodrug thereof, is in a mixture with the biocompatible polymer.

18. The drug delivery device according to claim 1, wherein the device consists essentially of the codrug, a pharmaceutically acceptable salt, or prodrug thereof.

19. The drug delivery device according to claim 1, wherein the first constituent moiety is the same as the second constituent moiety.

20. The drug delivery device according to claim 1, wherein the first constituent moiety is different from the second constituent moiety.

21. The drug delivery device according to claim 1, wherein the first and second constituent moieties are directly linked through a covalent bond formed between a functional group of the first constituent moiety and a functional group of the second constituent moiety.

22. The drug delivery device according to claim 1, wherein the first and second constituent moieties are linked to one another via a linking group that is covalently bonded to the first and second constituent moieties via functional groups thereon.

23. The drug delivery device according to claim 1, wherein the first constituent moiety is a corticosteroid.

24. The drug delivery device according to claim 1, wherein the second constituent moiety is a corticosteroid, an antiproliferative compound, or a non-steroidal anti-inflammatory compound.

25. The drug delivery device according to claim 1, wherein the corticosteroid is selected from triamcinolone acetonide, fluocinolone acetate, fluocinolone acetonide, cortisone, hydrocortisone, and hydrocortisone ester.

26. A method for treating a patient, comprising implanting a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device is implanted, injected or otherwise administered in the prostate, cervix, bladder, bladder neck, anal submucosa, or the tissues surrounding the aforementioned tissues or organs.

27. A method of inhibiting cell proliferation in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug, a pharmaceutically acceptable salt, or prodrug thereof.

28. A method of inhibiting inflammation in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

29. A method of reducing pain in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

30. The method of claim 29, wherein administering the device further comprises anesthetizing the patient.

31. A method of treating infection in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

32. The method of claim 31, wherein the infection is a viral, bacterial, or fungal infection.

33. A method of enhancing fertility in a patient in need of treatment, comprising implanting, injecting, or otherwise administering a drug delivery device comprising a codrug, or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

34. A method of improving sexual function in a patient in need of treatment, comprising implanting, injecting, or otherwise administering a drug delivery device comprising a codrug, or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

35. A method of reducing urinary urgency and/or frequency in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

36. A method of treating neovascularization in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

37. A method of treating muscle relaxation in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

38. A method of affecting localized immunomodulation in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

39. A method of treating incontinence in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

40. A method of treating tissue permeability changes in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

41. A method of treating hyperproliferation of tissue in a patient in need of treatment, comprising implanting, injecting, or otherwise administering a drug delivery device comprising a codrug or a pharmaceutically acceptable salt or prodrug thereof, for administration of at least one biologically active moiety, which codrug comprises:
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device includes a therapeutically effective amount of a codrug or a pharmaceutically acceptable salt or prodrug thereof.

42. The method according to claim 27, wherein the device is implanted, injected, or otherwise administered in the prostate, cervix, bladder, bladder neck, anal submucosa, or the tissues surrounding the aforementioned tissues or organs.

43. The method according to claim 26, wherein the genitourinary disorder is prostate cancer, prostatitis, cervical cancer, incontinence, a bladder disorder, benign prostatic hypertrophy (BPH), a chronic pelvic pain syndrome (e.g., irritable bowel syndrome, interstitial cystitis, prostatitis), uterine cancer, endometriosis, bladder cancer, sexual dysfunction (male and female), infertility, a sexually transmitted disease, or a urinary tract infection.

44. The method according to claim 27, further comprising radiation therapy, chemotherapy, transurethral resection of the prostrate, transurethral microwave therapy, transurethral thermal therapy, or laser ablation.

45. A kit comprising a drug delivery device of claim 1 in association with instructions (written and/or pictorial) describing the use of the device for treatment or prevention of a genitourinary disorder and optionally, warnings of possible side effects and drug-drug interactions.

46. A method of manufacturing a drug delivery device, comprising forming a codrug comprising
a) at least two constituent moieties, each moiety being a residue of a biologically active compound or a prodrug thereof, including a first constituent moiety and a second constituent moiety; and
b) a linkage covalently linking said at least two constituent moieties to form said codrug, said linkage is cleaved under physiological conditions to regenerate said constituent moieties;
wherein the device is dimensioned to position two radiation seeds a predetermined distance apart.
